(19) United States
(12) Patent Application Publication
(10) Pub. No.: US 2025/0043367 A1
(43) Pub. Date: Feb. 6, 2025

(54) METHODS FOR THE NON-INVASIVE DIAGNOSIS OF INTESTINAL INFECTION IN BIRDS

(71) Applicant: THE ROYAL VETERINARY COLLEGE, London (GB)

(72) Inventors: Jonathan WILLIAMS, London (GB); Damer BLAKE, London (GB)

(21) Appl. No.: 18/717,084

(22) PCT Filed: Dec. 5, 2022
(86) PCT No.: PCT/GB2022/053087

§ 371 Date: Jun. 6, 2024

(30) Foreign Application Priority Data
Dec. 6, 2021 (GB) 2117593.0

(51) Int. Cl. C12Q 1/6893

(52) U.S. Cl. CPC C12Q 1/6893; C12Q 2600/158; C12Q 2600/178

(57) ABSTRACT

The present invention provides means and methods for the non-invasive diagnosis of intestinal infection in birds.

### FIELD OF THE INVENTION

The invention is in the field of diagnostics.

### BACKGROUND

Coccidiosis is a serious intestinal disease in chickens (Gallus gallus domesticus) caused by protozoan parasites of the genus Eimeria, incurring significant morbidity and mortality, and economic losses (Blake, et al. 2020; Chapman, et al. 2013). Management and chemoprophylaxis remain the most important forms of anticoccidial control for broiler chickens, supplemented by a significant role for live vaccines in layer and breeding stock (Elwinger, et al. 2016). Unfortunately, resistance develops rapidly for every anticoccidial drug currently available and has become widespread, compromising economic productivity and animal welfare (Chapman 1999). Live wild-type (non-attenuated) and attenuated vaccines are highly effective and increasingly popular (Chapman and Jeffers 2014), but difficult to scale up for mainstream application in broiler production. Eimeria tenella is among the most common species to induce coccidiosis, is highly pathogenic and colonises the chicken caecum, causing haemorrhage, oedema, necrosis and anaemia (Györke et al., 2013).

Diagnosis of coccidiosis in broiler chickens and identification of causative Eimeria species is commonly achieved by post-mortem of dead or culled chickens, and/or euthanasia of a representative group of sentinel chickens. Other approaches to detect Eimeria infection include microscopic examination of faecal or litter samples to detect shed oocysts, although this is ineffective during the pre-patent period.

An alternative and simpler form of diagnosis to monitor and inform prevention, treatment and control methods would therefore be highly desirable.

### BRIEF SUMMARY OF THE INVENTION

The inventors have surprisingly found that not only are miRNAs stable in and recoverable from avian caecal content and faecal matter, but that faecal miRNAs reflect caecal content miRNAs, and are produced by the host and influenced by parasitic infection. A signature of miRNA markers that can be detected in the faecal content has been identified that correlates with the presence or absence of pathologically significant E. tenella infection and so can be used to diagnose a chicken or flock of chickens as infected with the parasite to a pathologically relevant level, or not-infected to a pathologically significant level, without having to euthanise the animal.

This ability to detect E. tenella infection and quantify the severity of disease offers value to routine flock surveillance, with specific applications in resource-intensive processes such as in vivo anticoccidial susceptibility testing (AST) (Peek and Landman 2003).

miRNA can be extracted from a wide range of tissues and bodily fluids, using a variety of commercially available kits. Different methods of extraction, including phenol-based, column-based and combinations of these can be used (REFS). Two such extraction kits are the ‘Norgen Stool RNA Purification Kit’ and the ‘MirVana miRNA Isolation Kit’. This study aimed to test the hypotheses that the stability of miRNA will enable its extraction and amplification from chicken caecal content, and that variation in a subset of these miRNAs produced by the host associates with the presence/absence and severity of E. tenella infection.

### DETAILED DESCRIPTION OF THE INVENTION

The inventors have surprisingly found that it is possible to identify a miRNA signature indicative of a pathologically significant level of intestinal infection in birds that is present in the faeces and caecal content of birds. The development of such a non-invasive method to determine whether a population of birds has a pathologically significant level of intestinal infection is highly advantageous for modern farming practices.

Although this approach has been exemplified in the context of Eimeria tenella infection, now that the skilled person knows it is possible to detect a relevant miRNA signature in the faeces of birds, the skilled person can apply the methodology described herein to other intestinal infections.

Since the inventors have found it to be possible to detect a signature of avian miRNAs in the faeces that correlates with an intestinal infection state, it is expected that similar miRNA profiles in response to other intestinal infections will be detectable in the faeces, for example in response to bacterial infections or infections with other intestinal parasites.

Accordingly in a first aspect the invention provides a method for determining a miRNA signature predictive of intestinal infection with an infectious agent in birds, wherein the method comprises:
a) providing at least:
   a first plurality of faecal and/or caecal samples; and
   a second plurality of faecal and/or caecal samples
wherein each of the faecal and/or caecal samples in the first plurality of faecal and/or caecal samples is taken from a different individual bird from a first population of birds that display a first infection phenotype and wherein each of the faecal and/or caecal samples in the second plurality of faecal and/or caecal samples is taken from a different individual bird from a second population of birds that display a second infection phenotype;
b) determining the relative amounts of a range of miRNAs in the first plurality of faecal and/or caecal samples and in the second plurality of faecal and/or caecal samples;
c) identifying miRNAs that are present in statistically different amounts between the first plurality of faecal and/or caecal samples level and the second plurality of faecal and/or caecal samples, and thereby identifying the miRNA signature predictive of infection with the infectious agent, optionally predictive of a pathologically significant intestinal infection.

A plurality of faecal and/or caecal samples can comprise any number of faecal and/or caecal samples. The skilled person will appreciate that preferably each faecal and/or caecal sample is taken from a different bird in the first and/or second population. In some instances multiple samples may be taken or derived from the same bird, but only in the context of multiple samples being taken from a range of different individual birds in the first and/or second population. The skilled person is able to select an appropriate number of individual birds in a given population so as to provide a robust statistically sound output. For example, in one embodiment the plurality of samples taken from or derived from at least 3, 4, 5, 6, 7, 8, 9, 10, 15, 20, 25, 30 or more different birds in a given population.

In this aspect of the invention in which the miRNA signature is established, it is preferable if each of the plurality of samples are from individual birds, that have a known phenotype. Once the miRNA signature has been established, in use it can be more practical to use a sample that comprises or may comprise faeces from a number of individuals, for example for routine screening purposes.

The skilled person will also appreciate that once a signature of miRNAs has been obtained that is predictive of infection with the infectious agent, for example predictive of a pathologically significant intestinal infection that requires treatment, the robustness of the miRNA signature should be tested against a further set of independent samples to corroborate the predictive power of the signature.

The miRNAs that are associated with the presence of intestinal infection caused by an infectious agent are typically derived from the test subject i.e., the bird, rather than being derived from the infectious agent, for example from a parasite or bacteria. Accordingly, it is considered that the response of the test bird to the presence of the infectious agent, for example the parasite, for example to the presence of a pathologically significant infection with the infectious agent/parasite, is responsible for the differential miRNA expression, rather than the differential miRNAs being expressed by the infectious agent itself.

In other embodiments one or more of the miRNAs that are associated with the presence of the infectious agent, for example with the presence of a pathologically significant infection with the infectious agent are derived from the infectious agent—i.e., are expressed by the parasite or bacteria. However, in preferred embodiments the one or more miRNAs that are associated with the presence of the infectious agent, for example with the presence of a pathologically significant infection with a parasite are derived from the test bird.

Attempts have previously been made to determine miRNA signatures in samples of the intestinal tissue of birds. This is clearly an invasive procedure and requires culling of one or more individuals. Prior to the present invention it was not considered possible to detect a predictive miRNA profile in faecal samples. However, the inventors have shown that this is possible, and that the miRNAs in the faecal sample largely correspond with the miRNAs that are found in the caecal matter.

Identifying miRNAs in faecal and/or caecal matter that are associated with intestinal infection in a bird can be performed by any appropriate method. The skilled person is well aware of suitable methods by which a biomarker signature can be derived for a particular disease or disease state. Such methods typically comprise determining the level of a selection of miRNAs (or even all miRNAs in some instance, for example where all of the miRNAs are sequenced, as described in the Examples) in two different sample types, e.g., diseased vs non-diseased, and analysis and comparison of the resultant expression levels between the different populations. Such methods are described in the examples, see for example, Example 7.

For example, for a given intestinal infection, the skilled person would determine the expression level of a set of miRNAs in a number of faecal samples from uninfected subjects and a number of faecal samples from infected subjects (or for example from different populations that may have different severities of infection). Commercial kits, equipment and computational programmes are available for such purposes. miRNAs that show statistically significant differential expression between the different populations (e.g., no infection vs infected; or no, low and high levels of infection).

In some embodiments the method comprises more than 2 populations, for example may comprise 3, or 4 or more populations, each with a different infection phenotype. For example, in some embodiments the method uses a first population with an infection phenotype of “uninfected”; a second population with an infection phenotype of “low level of infection”; and a third population with an infection phenotype of “high level of infection”.

Determining the amounts of all of or a range of miRNAs can be performed by any suitable means. For example, in a preferred embodiment, all miRNAs present in a sample are sequenced. In other embodiments, the miRNAs present in a sample may be hybridised to an array of probes.

In instances where all or a range of miRNAs are sequenced, it is possible to map the sequence reads to the genome of the test subject and/or the infectious agent, to determine potential genes that the miRNAs may silence.

The skilled person will appreciate that miRNAs are transcribed into a two strand (called a 5p and a 3p strand) stem loop structure which bind together. While bound they are effectively silenced until one or other strand is degraded by Dicer. Once the other strand is freed, it can then go on to alter gene expression. miRNA nomenclature therefore generally includes a 5p or a 3p to indicate the strand.

When mapping the miRNA sequences to the test subject or infectious agent genome, it is possible that there will be, in some cases, less than 100% sequence identity between the miRNA sequence reads and the reference genome of the test subject or infectious agents. Reference genomes are typically a single genome that is publicly available. However, polymorphisms will occur between individual test subjects and so it is expected that some miRNA sequences will not map perfectly to a reference genome.

Accordingly, although sequences of particular miRNAs may be identified as indicative of the presence or absence of infection, it is reasonable to expect there to be some divergence in these sequences across individuals. Accordingly, in some embodiments once a miRNA signature predictive of infection with the infectious agent has been derived using the method of the invention, putting that miRNA signature into effect requires detection of miRNAs that may have some sequence divergence from the sequences obtained for the miRNA signature.

In preferred embodiments the miRNAs are miRNAs that are derived from the subject, rather than from the infectious agent. Accordingly, in some embodiments, the miRNAs that are present in statistically different amounts between the first population of faecal and/or caecal samples level and the second population of faecal and/or caecal samples are mapped against the subject genome to identify those miRNAs that are derived from the subject, rather than the infectious agent.

The infectious agent may be an intestinal parasite or may be one or more bacteria or viruses. The infection may be driven by a co-infection of 2 different infectious agents, for example co-infection of a bacteria and a parasite.

In some embodiments the infectious agent is an avian parasite, for example is of the Eimerella species, for example is selected from Eimeria Tenella, Eimeria necatrix, Eimeria acervuline, Eimeria brunetti, Eimeria maxima, Eimeria mitis, Eimeria praecox. In preferred embodiments the infectious agent is Eimeria tenella or Eimeria necatrix.

In some embodiments the infectious agent is a bacteria, optionally is Salmonella sp. or E. coli.

In some embodiments the miRNA signature predictive of intestinal infection with an infectious agent is predictive of the presence of a pathologically significant level of intestinal infection. The skilled person will appreciate that there are many species, for example some parasites or bacteria, that have the potential to be pathogenic, but that are also present in the microflora of an organism without being pathologically relevant. For example, some species may be present in a subject at low levels, or in an inactive or non-pathogenic form. Detection of these infectious agents and treatment for infection may therefore be unnecessary in some instances. Preferably, a miRNA signature of the invention is able to distinguish between cases of pathologically relevant infection that requires treatment or other prophylactic measures to prevent infection of other subjects versus non-pathologically relevant infection, or non-infection.

For example, the presence of Eimeria sp. is common in chickens, but does not necessarily mean that the chicken or flock requires treatment—i.e. it is not necessarily a pathologically significant infection.

Once the miRNA signature has been obtained, it is then possible to use the signature to determine the presence of infection, or of a pathologically significant level of infection in a subject or population of a subjects, using a sample of the faecal and/or caecal matter.

Accordingly, the invention also provides a miRNA signature predictive of intestinal infection with an infectious agent in birds, optionally predictive of a pathologically significant intestinal infection or pathologically significant intestinal infection that requires treatment, wherein the signature has been derived using the method for determining a miRNA signature predictive of intestinal infection with an infectious agent in birds, optionally predictive of a pathologically significant intestinal infection or pathologically significant intestinal infection that requires treatment, of the invention. Preferences for features of this aspect of the invention are as described elsewhere herein, for example the infectious agent may be a parasite such as Eimeria sp., or may be a bacteria.

The invention also provides the use of the miRNA signatures described herein and that may be obtained using any of the methods described herein, to determine the presence of intestinal infection with an infectious agent in birds, optionally predictive of a pathologically significant intestinal infection or pathologically significant intestinal infection that requires treatment, wherein the miRNA signature if found in the faecal matter of caecal contents. Preferences for features of this aspect of the invention are as described elsewhere herein, for example the infectious agent may be a parasite such as Eimeria sp., or may be a bacteria.

The invention also provides a method for determining the presence of an intestinal infection with an infectious agent in one or more test birds, wherein the method comprises determining the level of one or more miRNAs in a sample of the faecal matter and/or caecal content from the one or more test birds, wherein the one or more miRNAs is associated with the presence of said infectious agent. Preferences for features of this aspect of the invention are as described elsewhere herein, for example the infectious agent may be a parasite such as Eimeria sp., or may be a bacteria.

The sample of faecal matter may be faecal matter from a single individual. Alternatively, the sample of faecal matter may be a sample that comprises faecal matter from a number of birds. For example, a sample of faecal matter from several subjects may be individually collected and mixed together to provide a single sample of faecal matter that comprises matter from several individuals. A more practical sample of faecal matter is a sample of faecal matter taken from the housing in which the birds are housed. For example, faecal matter may be taken from the flooring of the housing. Such samples may be used in the method of the invention individually or may be combined to provide a single sample.

Obtaining caecal matter requires that the bird is culled. Such a method is still considered to be useful, though preferably the sample is a sample of faecal matter that is obtained non-invasively and which does not require the culling or one or more subjects.

The infectious agent can be any avian infectious agent. In preferred embodiments the infectious agent is an avian intestinal parasite. In some embodiments the avian intestinal parasite is of the Eimeria species, i.e. is
a) Eimeralla sp;
b) selected from Eimeria Tenella, Eimeria necatrix, Eimeria acervuline, Eimeria brunetti, Eumeria maxima, Eimeria mitis, Eimeria praecox;
c) is Eimeria tenella or Eimeria necatrix; or
d) Eimeria tenella.

Preferably the avian intestinal parasite is Eimeria tenella.

In some embodiment the avian infectious agent is a bacteria that infects the intestines. For example, in some embodiments the infectious agent is a Salmonella sp. or E. coli.

As described above, it is considered to be useful if the method for determining the presence of an intestinal infection with an infectious agent is able to distinguish between those subjects or population of subjects that have a pathologically significant infection, for example a pathologically significant infection that requires treatment, and those that do not.

The terms “active”, “pathologically significant” and “pathologically significant infection that requires treatment” are used interchangeably throughout. The intention is to indicate a disease state that can be detected, and which warrants treatment. A method that detects the presence of an infectious agent but gives not information regarding whether the infection requires treatment is not considered to be as useful as a method that can distinguish between birds or populations of birds that require treatment, and those that don't but that may still carry a low level of the infectious agent.

By pathologically significant infection we include the meaning that the particular infectious agent is actively causing disease in the subject, for example is actively causing disease in the subject to an extent where treatment is required. For example, symptoms of a pathologically significant disease may include lethargy, anaemia, intestinal haemorrhage, weight loss and diarrhoea.

In some embodiments the method for determining the presence of an intestinal infection is a method for determining the presence of a pathologically significant level of intestinal infection. The skilled person will appreciate that there are many species, for example parasites or bacteria that have the potential to be pathogenic, but that are present in the microflora of an organism without being pathologically relevant. For example, some species may be present in a subject at low levels, or in an inactive or non-pathogenic form. Detection of these infectious agents and treatment for infection may therefore be unnecessary in some instances. Preferably, a method of the invention is able to detect cases of pathologically relevant infection that requires treatment or other prophylactic measures to prevent infection of other subjects.

For example, the presence of Eimeria sp., is common in chickens, but does not necessarily mean that the chicken or flock requires treatment.

In some embodiments, where the infectious agent is an intestinal parasite that causes lesions, for example where the infectious agent is Eimeria sp, for example is Eimeria tenella, pathologically significant infection is considered to occur when the lesion score is high. A well-known method of scoring lesions caused by Eimeria is described in Johnson and Reid 1970, and requires examination of samples of the upper, middle and lower intestine.

The method of Johnson and Reid 1970 scores lesions on a scale of 0 to +4, i.e. a particular subject is given a score of 0, 1, 2, 3 or 4. As described herein, a pathologically significant infection is present when the lesion score is above 0. Accordingly in one embodiment where the infectious agent is an intestinal parasite, for example is Eimeria sp., for example is Eimeria tenella, a pathologically significant infection is considered to occur when the lesion score is 1-4. In one embodiment the miRNA signature is able to discriminate between faecal or caecal samples have been derived from subjects that have a “low” lesion score of 1-2 and those that have a “high” lesion score of 3 or 4, i.e., a low-burden/less severe infection and those that have a high burden/more severity or pathologically relevant infection with the parasite. In one embodiment the method for determining the presence of an intestinal infection is a method for determining whether a subject has a low-burden/less severe infection and those that have a high burden/more severity or pathologically relevant infection with the parasite. In one embodiment the method for determining the presence of an intestinal infection is a method for determining whether a subject has a lesion score of 1-2 rather than 3-4.

The person skilled in this field will be well aware of how to score lesions, and what is considered to be a high lesion score.

It will be clear to the skilled person that when determining whether a level of one or more miRNAs indicates the presence of infection or not, the level of the one or more miRNAs in the sample from the test subject can be compared to a control value.

The control value can be a negative control, and/or can be a positive control. The skilled person is able to determine adequate control samples.

For example, in some embodiments the level of the one or more miRNAs in the test sample is compared to the level of the same one or more miRNAs in one or more negative control samples. A negative control sample may be, for example, the average level of the miRNA in faecal and/or caecal matter obtained from a number of subjects that are known to not be infected with the infectious agent. In some embodiments where the level of the one or more miRNAs in the sample from the test subject is higher or lower than the level in the control samples, the test subject is confirmed as being infected with the infectious agent, for example having a pathologically significant infection. In some embodiments the level of the miRNAs in the test subject has to be above, below or within pre-determined threshold or range for a determination of infection or pathologically significant infection to be made. Determining appropriate thresholds is within the skilled person's abilities.

Conversely, if the level of the one or more miRNAs in the test sample is the same as, or similar to the level of the miRNAs in the negative control samples, for example within a predetermined range, above or below a predetermined threshold, the subject is determined to not be infected, or to not be infected with a pathologically significant infection.

In some embodiments the control sample is a positive control sample, for example is the average level of the miRNA in faecal and/or caecal matter obtained from a number of subjects that are known to be infected with the infectious agent, for example known to be infected with a pathologically significant infection.

In some embodiments where the level of the one or more miRNAs in the sample from the test subject is the same as or higher than the level in the positive control samples or is within a pre-determined range, the test subject is confirmed as being infected with the infectious agent, for example having a pathologically significant infection. In some embodiments the level of the miRNAs in the test subject has to be above, below or within pre-determined threshold or range in order for a determination of infection or pathologically significant infection to be made. Determining appropriate thresholds is within the skilled person's abilities.

Conversely, if the level of the one or more miRNAs in the test sample is above or below the level of the miRNAs in the positive control samples, or above/below a predetermined threshold value or range, the subject is determined to not be infected, or to not be infected with a pathologically significant infection.

The control samples may be physical samples that are processed at the same time as the test sample. However it is generally more practical if the control samples are levels of each particular miRNA that have been predetermined, for example when developing the initial signature. In this way, the value of a given miRNA in the test sample can be compared to a known level of the same miRNA in a positive and/or negative control sample, and a determination made as to the presence or absence of infection.

The above is described in the context of the miRNAs that provide a signature that correlates with disease being over expressed in the diseased phenotype vs the non-diseased phenotype. The skilled person will however appreciate that there may be some miRNAs that are repressed in the diseased phenotype vs the non-diseased phenotype, and the skilled person will understand how to apply that signature, for example will know which controls to use and what comparisons to make.

In some embodiments the level of the one or more miRNAs determined in a test sample from the test subject is compared to both a positive and a negative control.

The present method is considered to be particularly advantageous for use in birds, not least because birds are often housed close together and detection of a pathologically significant infection is particularly important.

In more preferred embodiments the bird is a chicken (Gallus gallus) or a turkey. In particularly preferred embodiments the subject is a chicken or turkey that is housed in a flock, for example in a shed, pen, field or other enclosure sharing common space.

As mentioned above in relation to the method of determining a miRNA signature, preferably the miRNAs are produced by the test subject, for example are produced by the intestinal tissue of the test subject, rather than being produced by the infectious agent itself.

In some embodiments the one or more miRNAs that are associated with the presence of said infectious agent targets one or more genes selected from:
genes associated with the Mucin type O-Glycan biosynthesis pathway

In some particular embodiments, the one or more miRNAs that are associated with the presence of said infectious agent targets any one or more of the following genes: GALNT16, GALNT6, GALNT12, GALNT14, GALNT5 and GCNT4. The skilled person will appreciate that many different miRNAs may target the same gene.

As described above, for any miRNA signature indicative of infection/absence of infection, it is likely that there will be some polymorphisms in the miRNAs expressed by different individuals. Accordingly, in some embodiments the method for determining the presence of an intestinal infection with an avian infectious agent in one or more test subjects, comprises determining the level of one or more miRNAs in a sample of the faecal matter and/or caecal content from the one or more test subjects, wherein the one or more miRNAs has a sequence with at least 90%, 91%, 92%, 93%, 94%, 95%, 96%, 97%, 98%, 99% or 100% sequence identity to a reference miRNA sequence.

The miRNA reference sequence is the sequence that was obtained when deriving the miRNA signature, for example using a method according to the first aspect of the invention. i.e., a particular miRNA that is identified as being differentially expressed between the at least two populations (e.g., infected and non-infected) is in some embodiments considered to be a reference sequence, and in practice, when using the miRNA signature, the skilled person determines the level of miRNAs in the test sample that have at least 90%, 91%, 92%, 93%, 94%, 95%, 96%, 97%, 98%, 99% or 100% sequence identity to the one or reference miRNA sequences that make up the miRNA signature.

Accordingly, although sequences of particular miRNAs may be identified as indicative of the presence or absence of infection, it is reasonable to expect there to be some divergence in these sequences across individuals. Accordingly, in some embodiments once a miRNA signature predictive of infection with the infectious agent has been derived using the method of the invention, putting that miRNA signature into effect requires detection of miRNAs that may have some sequence divergence from the sequences obtained for the miRNA signature.

In one embodiment, miRNAs that are indicative of the presence of infection with Eimeria sp., for example Eimeria tenella can comprise or consist of any one or more of, for example any 1, 2, 3, 4, 5, 6, 7, 8, 9, 10, 11, 12, 13, 14, 15, 16, 17, 18, or 19 or more or all of the following miRNAs:

[Full list of the relevant miRNAs (* indicates there is sequence divergence from miRbase ID (https://mirbase.org/):

| | | | Up or |
|---|---|---|---|
| SEQ ID | miRbase ID | Sequence | down |
| [SEQ ID NO: 1] | gga-miR-193b-3p* | AACTGGCCCACAAAGTCCCGCT | Up |
| [SEQ ID NO: 2] | gga-miR-2188-5p | AAGGTCCAACCTCACATGTCCT | Up |
| [SEQ ID NO: 3] | gga-miR-140-3p* | ACCACAGGGTAGAACCACGGACT | Up |
| [SEQ ID NO: 4] | gga-miR-146c-5p* | TGAGAACTGAATTCCATGGACT | Up |
| [SEQ ID NO: 5] | gga-miR-19a-3p* | TGTGCAAATCCATGCAAAACTGA | Up |
| [SEQ ID NO: 6] | tgu-miR-425-5p* | AATGACACGATCACTCCCGCTGAGC | Up |
| [SEQ ID NO: 7] | gga-miR-22-3p | AAGCTGCCAGTTGAAGAACTGT | Up |
| [SEQ ID NO: 8] | gga-miR-7* | TGGAAGACTAGTGATTTTGTTGTT | Up |
| [SEQ ID NO: 9] | gga-let-7c-5p* | TGAGGTAGTAGGTTGTATAGTTT | Down |
| [SEQ ID NO: 10] | gga-miR-142-5p | CCCATAAAGTAGAAAGCACTAC | Up |
| [SEQ ID NO: 11] | gga-miR-23b-3p* | ATCACATTGCCAGGGATTTCC | Up |
| [SEQ ID NO: 12] | gga-miR-7b* | TGGAAGACTAGTGATTTTTGTTGT | Up |
| [SEQ ID NO: 13] | gga-miR-30b-5p | TGTAAACATCCTACACTCAGCT | Down |
| [SEQ ID NO: 14] | gga-miR-219b | CACAAGAATTGCGTTTGGACAA | Up |
| [SEQ ID NO: 15] | tgu-miR-363-3p | AATTGCACGGTATCCATCTGT | Up |
| [SEQ ID NO: 16] | gga-miR-128-3p | TCACAGTGAACCGGTCTCTTT | Up |
| [SEQ ID NO: 17] | gga-miR-19b-3p* | TGTGCAAATCCATGCAAAACTGT | Up |
| [SEQ ID NO: 18] | gga-miR-101-3p* | GTACAGTACTGTGATAACTGA | Up |
| [SEQ ID NO: 19] | gga-miR-183* | TATGGCACTGGTAGAATTCACT | Down |

The table above describes the direction of differential regulation with respect to the expression level of the miRNA in an uninfected control bird, or uninfected population of control birds. For example, 3 or the miRNAs above were found to be associated with infection with Eimeria sp, for example Eimeria tenella when the expression level of the marker was lower, or downregulated, with respect to a control bird or control population. 16 of the above miRNAs were found to be associated with infection with Eimeria sp, for example Eimeria tenella when the expression level of the marker was higher, or upregulated, with respect to a control bird or control population.

As is clear from the data presented in the Examples, for example FIG. 4, each individual miRNA described above is considered to have sufficient predictive power alone to predict infection, for example infection with Eimeria sp., for example Eimeria tenella.

The direction of differential expression associated with infection for each of the miRNAs described in the above table applies throughout.

In one embodiment, the miRNAs that are indicative of the presence of infection with Eimeria sp, for example Eimeria tenella can comprise or consist of any one or more of, for example any 1, 2, 3, 4, 5, 6, 7, or 8 or all of the following miRNAs:

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

Direction of differential expression associated with infection is as described in the table above.

In one embodiment the miRNAs that are indicative of the presence of infection with Eimeria sp., for example Eimeria tenella can comprise or consist of any one or more of, for example any 1, 2, 3, 4 or 5 of:

| [SEQ ID NO: 2] | |
|---|---|
| gga-miR-2188-5p | AAGGTCCAACCTCACATGTCCT |
| [SEQ ID NO: 4] | |
| gga-miR-146c-5p* | TGAGAACTGAATTCCATGGACT |
| [SEQ ID NO: 5] | |
| gga-miR-19a-3p* | TGTGCAAATCCATGCAAAACTGA |
| [SEQ ID NO: 7] | |
| gga-miR-22-3p | AAGCTGCCAGTTGAAGAACTGT |
| [SEQ ID NO: 8] | |
| gga-miR-7* | TGGAAGACTAGTGATTTTGTTGTT |

Direction of differential expression associated with infection is as described in the table above.

In one embodiment the miRNAs that are indicative of the presence of infection with Eimeria sp., for example Eimeria tenella can comprise or consist of any one or more of, for example any 1, 2 or 3 of:

| [SEQ ID NO: 2] | |
|---|---|
| gga-miR-2188-5p | AAGGTCCAACCTCACATGTCCT |
| [SEQ ID NO: 5] | |
| gga-miR-19a-3p* | TGTGCAAATCCATGCAAAACTGA |
| [SEQ ID NO: 7] | |
| gga-miR-22-3p | AAGCTGCCAGTTGAAGAACTGT |

Direction of differential expression associated with infection is as described in the table above.

For example in some preferred embodiments where the sample is a sample of faecal matter, the miRNAs that are indicative of the presence of infection with Eimeria sp., for example Eimeria tenella can comprise or consist of any one or more of, for example any 1, 2 or 3 of:

| [SEQ ID NO: 2] | |
|---|---|
| gga-miR-2188-5p | AAGGTCCAACCTCACATGTCCT |
| [SEQ ID NO: 5] | |
| gga-miR-19a-3p* | TGTGCAAATCCATGCAAAACTGA |
| [SEQ ID NO: 7] | |
| gga-miR-22-3p | AAGCTGCCAGTTGAAGAACTGT |

Direction of differential expression associated with infection is as described in the table above.

In one embodiment the miRNAs that are indicative of the presence of infection with Eimeria sp., for example Eimeria tenella can comprise or consist of any one or more of, for example any 1, 2, 3, 4, 5, 6, 7, 8, 9, 10, 11, 12, 13, 14, 15, 16, 17, 18, or 19 or all of the miRNAs of [SEQ ID NO: 1]-[SEQ ID NO: 19] or of a sequence that has at least 90%, 91%, 92%, 93%, 94%, 95%, 96%, 97%, 98%, 99% or 100% sequence identity to the one or more miRNAs of [SEQ ID NO: 1]-[SEQ ID NO: 19].

In one embodiment the miRNAs that are indicative of the presence of infection with Eimeria sp., for example Eimeria tenella can comprise or consist of any one or more of, any 1, 2, 3, 4, 5, 6, 7, or 8 or all of the miRNAs of [SEQ ID NO: 1]-[SEQ ID NO: 8] or of a sequence that has at least 90%, 91%, 92%, 93%, 94%, 95%, 96%, 97%, 98%, 99% or 100% sequence identity to the one or more miRNAs of [SEQ ID NO: 1]-[SEQ ID NO: 8].

In one embodiment the miRNAs that are indicative of the presence of infection with Eimeria sp., for example Eimeria tenella can comprise or consist of any one or more of, for example any 1, 2, 3, 4, or 5 or all of the miRNAs of [SEQ ID NO: 2, 4, 5, 7, or 8] or of a sequence that has at least 90%, 91%, 92%, 93%, 94%, 95%, 96%, 97%, 98%, 99% or 100% sequence identity to the one or more miRNAs of [SEQ ID NO: 2, 4, 5, 7, or 8].

In one embodiment the miRNAs that are indicative of the presence of infection with Eimeria sp., for example Eimeria tenella can comprise or consist of any one or more of, for example any 1, 2, or 3 or all of the miRNAs of [SEQ ID NO: 2, 5 or 7] or of a sequence that has at least 90%, 91%, 92%, 93%, 94%, 95%, 96%, 97%, 98%, 99% or 100% sequence identity to the one or more miRNAs of [SEQ ID NO: 2, 5 or 7].

The level of the one or more miRNAs in the sample can be determined using any suitable means. For example, in one embodiment the levels of the miRNAs are determined using reverse transcription followed by qPCR.

The skilled person is able to design appropriate primers to amplify the required miRNA(s). For example, when detecting the following miRNAs, the following primers may be used:

TABLE 1
Sequences of selected miRNAs identified in this study and used as primers
for validation qPCR. Note, the reverse primer used in validation for all target miRNAs
was the Universal mRQ 3′ Primer supplied with the Mir-X™ miRNA First-Strand
Synthesis and SYBR® qRT-PCR kit. cel-miR-39 represents a non-target sequence from
C. Elegans used as a negative control.

| | | Reverse primer |
|---|---|---|
| | | (Universal mRQ 3′ |
| | | Primer supplied Mir- |
| | | X™ miRNA First- |
| | | Strand Synthesis and |
| miRNA | Forward primer | SYBR® qRT-PCR kit) |
| [SEQ ID NO: 1] gga- | AAGGTCCAACCTCACATGTCCT | mRQ 3′ Primer |
| miR-2188-5p | [SEQ ID NO: 20] | |
| [SEQ ID NO: 2] | TGTGCAAATCCATGCAAAACTGA | mRQ 3′ Primer |
| gga19a-3p* | [SEQ ID NO: 21] | |
| [SEQ ID NO: 3] gga- | AAGCTGCCAGTTGAAGAACTGT | mRQ 3′ Primer |
| miR-22-3p | [SEQ ID NO: 22] | |
| [SEQ ID NO: 4] gga7* | TGGAAGACTAGTGATTTTGTTGTT | mRQ 3′ Primer |
| | [SEQ ID NO: 23] | |
| [SEQ ID NO: 5] | TGAGAACTGAATTCCATGGACT | mRQ 3′ Primer |
| gga146c-5p* | [SEQ ID NO: 24] | |
| [SEQ ID NO: 6] | AACTGGCCCACAAAGTCCCGCT | mRQ 3′ Primer |
| gga193b-3p* | [SEQ ID NO: 25] | |
| [SEQ ID NO: 7] | ACCACAGGGTAGAACCACGGACT | mRQ 3′ Primer |
| gga140-3p* | [SEQ ID NO: 26] | |
| [SEQ ID NO: 8] | AATGACACGATCACTCCCGCTGAGC | mRQ 3′ Primer |
| tgu425-5p* | [SEQ ID NO: 27] | |

The reverse primer described in the above table is taken from the Takara miscript kit. The skilled person will realise that is possible to amplify each of the above miRNAs either with a unique forward and reverse primer pair per miRNA, or with a further common reverse primer.

In some other embodiments, determination of the level of the one or more miRNAs is via hybridisation of miRNA to a detection probe. The detection probe may be an oligonucleotide for example.

Preferably the method of determining the level of the one or more miRNAs produces a visible readout, i.e., visible to the naked eye, so that the method can be performed in the absence of sophisticated laboratory equipment.

Any suitable method can be used to obtain RNA from the faecal and/or caecal sample. The inventors have demonstrated that two commercially available kits (Cat. No. AM 1560, Lot 00360891, miRvana, ThermoFisher Scientific, Massachusetts; and Norgen Stool Total RNA Purification Kit Cat. No. 49500, Biotek, Canada) are suitable for extracting RNA from avian faecal and caecal samples.

The inventors have surprisingly found that, contrary to prior art methods of extracting RNA from fecal and caecal samples that require the isolation or purification of membrane vesicles such as microvesicles and/or exosomes from the sample, it is possible to obtain sufficient and appropriate RNA from the sample using standard, rapid total-RNA extraction methods, such as the kits described above. These methods are simpler and quicker to perform since they do not require the step of isolating the membrane vesicles.

Accordingly, in some embodiments the method used to obtain RNA from the faecal and/or caecal sample does not require isolation of or purification of microvesicles, for example does not require the isolation of exosomes or other lipid structures that comprise RNA. Methods of isolation of membrane vesicles such as microvesicles and exosomes can include a) ultracentrifugation for example spinning at 10,000 g or more for 1-3 hours; or b) ultrafiltration for example using 100 kDa filters. Other methods of isolating or purifying membrane vesicles such as microvesicles and exosomes include differential centrifugation, ion exchange and/or gel permeation chromatography, sucrose density gradients, organelle electrophoresis and other methods.

Accordingly in some embodiments the invention provides a method for determining the presence of an intestinal infection in one or more test birds wherein the intestinal infection is caused by an infectious agent,
wherein the method comprises determining the level of one or more miRNAs in a faecal and/or caecal sample obtained from the one or more test birds, wherein the level of the one or more miRNAs represents a miRNA signature indicative of the presence of said intestinal infection, wherein determining the level of one or more miRNAs in the sample does not require isolation or purification or membrane vesicles, such as microvesicles or exosomes.

In some embodiments, the methods described herein further comprise, upon the determination of the presence of infection with the avian intestinal parasite, for example upon identification of a pathologically significant infection, the subject or population of subjects is treated with a therapeutic to treat infection with the avian intestinal parasite.

In the same or other embodiments, upon the determination of the presence of infection with the avian intestinal parasite, for example upon identification of a pathologically significant infection, the subject or population of subjects are isolated from other non-infected subjects.

The invention also provides a method for determining the efficacy of a treatment against one or more intestinal infections in a bird wherein the method comprises:
a) determining the level of one or more miRNAs in a sample of the faeces and/or caecal content from the one or more test birds, wherein the one or more miRNAs represents a miRNA signature indicative of the presence of said intestinal infection, for example a pathologically significant intestinal infection or pathologically significant intestinal infection that requires treatment according to the method of the invention described herein;
b) determining the presence of said intestinal infection, for example pathologically significant intestinal infection or pathologically significant intestinal infection that requires treatment based upon the level of the one or more miRNAs determined in (a);
c) treating said bird with an appropriate therapeutic to treat said intestinal infection;
d) following the treatment in step (c), repeating step (a);
e) confirming the presence of said intestinal infection, for example pathologically significant intestinal infection or pathologically significant intestinal infection that requires treatment based upon the level of the one or more miRNAs determined in (d).

The invention also provides a method for determining the efficacy of a virulent vaccine against intestinal infection in a bird wherein the method comprises determining the presence of the intestinal infection or intestinal response to virulent vaccine, for example a pathologically significant intestinal infection according to the method of the invention, for example that requires treatment. In some embodiments the virulent vaccine is considered to have been effective if the presence of the intestinal infection is detected.

The invention also provides a method for detecting harmful effects of a virulent vaccine against intestinal infection in a bird wherein the method comprises:
a) determining the presence of said intestinal infection, or pathologically significant intestinal infection or pathologically significant intestinal infection that requires treatment according to any method of the invention at:
   i) a first time point; and
   ii) a second time point;
      wherein the first time point and second time point are selected so as to allow determination of persistent presence of the intestinal infection, wherein persistent presence of the intestinal infection indicates harmful effects of the virulent vaccine.

The invention also provides various methods of treating an infection with the infectious agent where a subject or population of subjects (i.e., bird or population of birds) is determined to be infected with the infectious agent, for example have a pathologically significant infection that requires treatment.

Accordingly, the invention provides an anti-avian intestinal infection therapeutic for use in treating an avian intestinal infection wherein the infection has been detected as requiring treatment using any of the methods of the invention. In some embodiments, as described elsewhere herein, the intestinal infection is an infection with a parasite or a bacteria or a virus, and in some preferred embodiments the parasite is Eimeria sp., for example Eimeria tenella.

The invention also provides a method for screening a population of birds, for example a population of chickens for the presence of an intestinal infection, the method comprises determining the presence of an intestinal infection using any of the methods of the invention, wherein the sample is a sample of faecal matter taken from the avian environment. In some embodiments, as described elsewhere herein, the intestinal infection is an infection with a parasite or a bacteria or a virus, and in some preferred embodiments the parasite is Eimeria sp., for example Eimeria tenella. In some embodiments the sample comprises a number of samples of faecal matter taken from the avian environment. In some embodiment the method for screening is performed at periodic intervals, optionally at intervals of 1 week, 2 weeks, 3 weeks, 4 weeks, 1 month, 2 months, 3 months, 4 months, 5 months or 6 months or more. In some embodiments the method comprises comparing the level of the one or more miRNAs in the sample between intervals to determine an elevation or depression in the level of the one or more miRNAs, wherein an increase in the level of the one or more miRNAs indicates an increased presence of an avian intestinal parasite in the population of birds for example chickens. In some embodiments if an increase in the level of the one or more miRNAs is detected, the population is treated with a therapeutic agent to treat the intestinal infection.

The invention also provides a kit for performing any of the methods described herein.

In one embodiment the kit comprises:
a) Means to determine the level of at least one of the miRNAs associated with the presence of said intestinal infection;
b) means for the extraction and purification of RNA from a caecal and/or faecal sample;
c) a control sample of RNA prepared from a faecal sample from subjects that are not infected with the intestinal infection.

In some embodiments the means to determine the level of at least one of the miRNAs comprises:
a) at least one pair of primers are arranged so as to amplify at least one or more or all of the following miRNAs:

i)
[SEQ ID NO: 1]

| | |
|---|---|
| gga-miR-193b-3p* | AACTGGCCCACAAAGTCCCGCT |

[SEQ ID NO: 2]

| | |
|---|---|
| gga-miR-2188-5p | AAGGTCCAACCTCACATGTCCT |

[SEQ ID NO: 3]

| | |
|---|---|
| gga-miR-140-3p* | ACCACAGGGTAGAACCACGGACT |

[SEQ ID NO: 4]

| | |
|---|---|
| gga-miR-146c-5p* | TGAGAACTGAATTCCATGGACT |

[SEQ ID NO: 5]

| | |
|---|---|
| gga-miR-19a-3p* | TGTGCAAATCCATGCAAAACTGA |

[SEQ ID NO: 6]

| | |
|---|---|
| tgu-miR-425-5p* | AATGACACGATCACTCCCGCTGAGC |

[SEQ ID NO: 7]

| | |
|---|---|
| gga-miR-22-3p | AAGCTGCCAGTTGAAGAACTGT |

[SEQ ID NO: 8]

| | |
|---|---|
| gga-miR-7* | TGGAAGACTAGTGATTTTGTTGTT |

[SEQ ID NO: 9]

| | |
|---|---|
| gga-let-7c-5p* | TGAGGTAGTAGGTTGTATAGTTT |

[SEQ ID NO: 10]

| | |
|---|---|
| gga-miR-142-5p | CCCATAAAGTAGAAAGCACTAC |

[SEQ ID NO: 11]

| | |
|---|---|
| gga-miR-23b-3p* | ATCACATTGCCAGGGATTTCC |

[SEQ ID NO: 12]

| | |
|---|---|
| gga-miR-7b* | TGGAAGACTAGTGATTTTTGTTGT |

[SEQ ID NO: 13]

| | |
|---|---|
| gga-miR-30b-5p | TGTAAACATCCTACACTCAGCT |

[SEQ ID NO: 14]

| | |
|---|---|
| gga-miR-219b | CACAAGAATTGCGTTTGGACAA |

[SEQ ID NO: 15]

| | |
|---|---|
| tgu-miR-363-3p | AATTGCACGGTATCCATCTGT |

[SEQ ID NO: 16]

| | |
|---|---|
| gga-miR-128-3p | TCACAGTGAACCGGTCTCTIT |

[SEQ ID NO: 17]

| | |
|---|---|
| gga-miR-19b-3p* | TGTGCAAATCCATGCAAAACTGT |

[SEQ ID NO: 18]

| | |
|---|---|
| gga-miR-101-3p* | GTACAGTACTGTGATAACTGA |

[SEQ ID NO: 19]

| | |
|---|---|
| gga-miR-183* | TATGGCACTGGTAGAATTCACT |

or
ii)
[SEQ ID NO: 1]

gga-miR-193b-3p*

[SEQ ID NO: 2]

gga-miR-2188-5p

[SEQ ID NO: 3]

gga-miR-140-3p*

[SEQ ID NO: 4]

gga-miR-146c-5p*

[SEQ ID NO: 5]

gga-miR-19a-3p*

[SEQ ID NO: 6]

tgu-miR-425-5p*

[SEQ ID NO: 7]

gga-miR-22-3p

[SEQ ID NO: 8]

gga-miR-7*

iii)
[SEQ ID NO: 2]

| | |
|---|---|
| gga-miR-2188-5p | AAGGTCCAACCTCACATGTCCT |

[SEQ ID NO: 4]

| | |
|---|---|
| gga-miR-146c-5p* | TGAGAACTGAATTCCATGGACT |

[SEQ ID NO: 5]

| | |
|---|---|
| gga-miR-19a-3p* | TGTGCAAATCCATGCAAAACTGA |

[SEQ ID NO: 7]

| | |
|---|---|
| gga-miR-22-3p | AAGCTGCCAGTTGAAGAACTGT |

[SEQ ID NO: 8]

| | |
|---|---|
| gga-miR-7* | TGGAAGACTAGTGATTTTGTTGTT; |

or
ivi)
[SEQ ID NO: 2]

| | |
|---|---|
| gga-miR-2188-5p | AAGGTCCAACCTCACATGTCCT |

[SEQ ID NO: 5]

| | |
|---|---|
| gga-miR-19a-3p* | TGTGCAAATCCATGCAAAACTGA |

[SEQ ID NO: 7]

| | |
|---|---|
| gga-miR-22-3p | AAGCTGCCAGTTGAAGAACTGT |

and/or
b) one or more hybridisation probes able to specifically hybridise to one or more of the following miRNAs:

i)
[SEQ ID NO: 1]

| | |
|---|---|
| gga-miR-193b-3p* | AACTGGCCCACAAAGTCCCGCT |

[SEQ ID NO: 2]

| | |
|---|---|
| gga-miR-2188-5p | AAGGTCCAACCTCACATGTCCT |

[SEQ ID NO: 3]

| | |
|---|---|
| gga-miR-140-3p* | ACCACAGGGTAGAACCACGGACT |

[SEQ ID NO: 4]

| | |
|---|---|
| gga-miR-146c-5p* | TGAGAACTGAATTCCATGGACT |

[SEQ ID NO: 5]

| | |
|---|---|
| gga-miR-19a-3p* | TGTGCAAATCCATGCAAAACTGA |

[SEQ ID NO: 6]

| | |
|---|---|
| tgu-miR-425-5p* | AATGACACGATCACTCCCGCTGAGC |

[SEQ ID NO: 7]

| | |
|---|---|
| gga-miR-22-3p | AAGCTGCCAGTTGAAGAACTGT |

[SEQ ID NO: 8]

| | |
|---|---|
| gga-miR-7* | TGGAAGACTAGTGATTTTGTTGTT |

[SEQ ID NO: 9]

| | |
|---|---|
| gga-let-7c-5p* | TGAGGTAGTAGGTTGTATAGTTT |

[SEQ ID NO: 10]

| | |
|---|---|
| gga-miR-142-5p | CCCATAAAGTAGAAAGCACTAC |

[SEQ ID NO: 11]

| | |
|---|---|
| gga-miR-23b-3p* | ATCACATTGCCAGGGATTTCC |

[SEQ ID NO: 12]

| | |
|---|---|
| gga-miR-7b* | TGGAAGACTAGTGATTTTTGTTGT |

[SEQ ID NO: 13]

| | |
|---|---|
| gga-miR-30b-5p | TGTAAACATCCTACACTCAGCT |

[SEQ ID NO: 14]

| | |
|---|---|
| gga-miR-219b | CACAAGAATTGCGTTTGGACAA |

[SEQ ID NO: 15]

| | |
|---|---|
| tgu-miR-363-3p | AATTGCACGGTATCCATCTGT |

[SEQ ID NO: 16]

| | |
|---|---|
| gga-miR-128-3p | TCACAGTGAACCGGTCTCTIT |

[SEQ ID NO: 17]

| | |
|---|---|
| gga-miR-19b-3p* | TGTGCAAATCCATGCAAAACTGT |

[SEQ ID NO: 18]

| | |
|---|---|
| gga-miR-101-3p* | GTACAGTACTGTGATAACTGA |

[SEQ ID NO: 19]

| | |
|---|---|
| gga-miR-183* | TATGGCACTGGTAGAATTCACT |

ii)
[SEQ ID NO: 1]

gga-miR-193b-3p*

[SEQ ID NO: 2]

gga-miR-2188-5p

[SEQ ID NO: 3]

gga-miR-140-3p*

[SEQ ID NO: 4]

gga-miR-146c-5p*

[SEQ ID NO: 5]

gga-miR-19a-3p*

[SEQ ID NO: 6]

tgu-miR-425-5p*

[SEQ ID NO: 7]

gga-miR-22-3p

[SEQ ID NO: 8]

gga-miR-7*

iii)
[SEQ ID NO: 2]

| | |
|---|---|
| gga-miR-2188-5p | AAGGTCCAACCTCACATGTCCT |

[SEQ ID NO: 4]

| | |
|---|---|
| gga-miR-146c-5p* | TGAGAACTGAATTCCATGGACT |

[SEQ ID NO: 5]

| | |
|---|---|
| gga-miR-19a-3p* | TGTGCAAATCCATGCAAAACTGA |

[SEQ ID NO: 7]

| | |
|---|---|
| gga-miR-22-3p | AAGCTGCCAGTTGAAGAACTGT |

[SEQ ID NO: 8]

| | |
|---|---|
| gga-miR-7* | TGGAAGACTAGTGATTTTGTTGTT; |

or
iv)
[SEQ ID NO: 2]

| | |
|---|---|
| gga-miR-2188-5p | AAGGTCCAACCTCACATGTCCT |

[SEQ ID NO: 5]

| | |
|---|---|
| gga-miR-19a-3p* | TGTGCAAATCCATGCAAAACTGA |

[SEQ ID NO: 7]

| | |
|---|---|
| gga-miR-22-3p | AAGCTGCCAGTTGAAGAACTGT |

Preferences and options for a given aspect, feature or parameter of the invention should, unless the context indicates otherwise, be regarded as having been disclosed in combination with any and all preferences and options for all other aspects, features and parameters of the invention. For example, the invention provides:

1. A method for determining a miRNA signature predictive of Salmonella infection in birds, wherein the method comprises:
a) providing at least:
   a first plurality of faecal and/or caecal samples; and
   a second plurality of faecal and/or caecal samples
wherein the first plurality of faecal and/or caecal samples is from a first population of birds that are infected with Salmonella and wherein the second plurality of faecal and/or caecal samples is from a second population of birds that are not infected with Salmonella;
b) determining the relative amounts of a range of miRNAs in the first plurality of faecal and/or caecal samples and in the second plurality of faecal and/or caecal samples;
c) identifying miRNAs that are present in statistically different amounts between the first plurality of faecal and/or caecal samples level and the second plurality of faecal and/or caecal samples, and thereby identifying the miRNA signature predictive of infection with Salmonella.

And the invention also provides:

2. A method for determining the presence of a pathologically significant intestinal infection with E. tenella
wherein the method comprises determining the level of one or more miRNAs in a faecal sample obtained from the one or more test birds, wherein the level of the one or more miRNAs represents a miRNA signature indicative of the presence of said intestinal infection, and comparing the level of the one or more miRNAs to the level of the same one or more miRNAs in a negative control sample, or to a pre-determined negative control value,
wherein the one or more miRNAs comprise any one, two or three of:

| [SEQ ID NO: 2] | |
|---|---|
| gga-miR-2188-5p | AAGGTCCAACCTCACATGTCCT |
| [SEQ ID NO: 5] | |
| gga-miR-19a-3p* | TGTGCAAATCCATGCAAAACTGA |
| [SEQ ID NO: 7] | |
| gga-miR-22-3p | AAGCTGCCAGTTGAAGAACTGT |

The listing or discussion of an apparently prior-published document in this specification should not necessarily be taken as an acknowledgement that the document is part of the state of the art or is common general knowledge.

The invention is also further defined by reference to the following numbered paragraphs:

1. A method for determining the presence of an intestinal infection in one or more test birds wherein the intestinal infection is caused by an infectious agent,
wherein the method comprises determining the level of one or more miRNAs in a faecal and/or caecal sample obtained from the one or more test birds, wherein the level of the one or more miRNAs represents a miRNA signature indicative of the presence of said intestinal infection.

2. A method for determining the presence of a pathologically significant intestinal infection or a pathologically significant intestinal infection that requires treatment in one or more test birds, wherein the intestinal infection is caused by an infectious agent,
wherein the method comprises determining the level of one or more miRNAs in a faecal and/or caecal sample obtained from the one or more test birds, wherein the level of the one or more miRNAs represents a miRNA signature indicative of the presence of said pathologically significant intestinal infection or pathologically significant intestinal infection that requires treatment.

3. The method according to paragraph 1 or 2 wherein the infectious agent is an avian intestinal parasite, optionally is:
a) Eimeralla sp;
b) selected from Eimeria Tenella, Eimeria necatrix, Eimeria acervuline, Eimeria brunetti, Eumeria maxima, Eimeria mitis, Eimeria praecox;
c) is Eimeria tenella or Eimeria necatrix; or
d) Eimeria tenella

4. The method according to paragraph 1 or 2 wherein the infectious agent is a bacteria, optionally is Salmonella sp. or E. coli.

5. The method according to any of paragraphs 1˜4 wherein the one or more test birds is a chicken or a turkey, optionally is a chicken (Gallus gallus).

6. The method according to any of paragraphs 1-5 wherein the one or more miRNAs are produced by the test bird, optionally produced by the intestinal tissue of the test bird.

7. The method according to any of the preceding paragraphs wherein the method further comprises comparing the level of the one or more miRNAs to the level of the same one or more miRNAs in one or more control samples.

8. The method according to paragraph 7 wherein the control sample is a negative control faecal and/or caecal sample obtained from one or more birds that are not infected with the infectious agent, or are not infected with a pathologically relevant infection or a pathologically significant intestinal infection that requires treatment,
and wherein the test bird is determined to be infected with the infectious agent optionally infected with a pathologically significant infection or a pathologically significant intestinal infection that requires treatment, if the level of the one or more miRNAs in the faecal and/or caecal sample from the test bird is:
a) higher than the level of the one or more miRNAs in the negative control sample; and/or
b) above a predetermined range or within a pre-determined threshold level.

9. The method according to paragraph 8 wherein the test bird is determined to not be infected or to not be infected with a pathologically significant infection or a pathologically significant intestinal infection that requires treatment where the level of the one or more miRNAs in the test sample is:
a) the same as or substantially similar to the level of the same miRNAs in the negative control sample; and/or
b) within a predetermined range or below a predetermined threshold.

10. The method according to paragraph 7-9 wherein the control sample is a positive control faecal and/or caecal sample obtained from one or more birds that are infected with the infectious agent, or are infected with a pathologically relevant infection or a pathologically significant intestinal infection that requires treatment,
and wherein the test bird is determined to be infected with the infectious agent optionally infected with a pathologically significant infection or a pathologically significant intestinal infection that requires treatment, if the level of the one or more miRNAs in the faecal and/or caecal sample from the test subject is:
a) the same as or higher than the level of the same one or more miRNAs in the positive control sample; and/or
b) within a predetermined range of the level of the one or more miRNAs in the positive control sample.

11. The method according to paragraph 7-10 wherein the test subject is determined to not be infected or to not be infected with a pathologically significant infection or a pathologically significant intestinal infection that requires treatment where the level of the one or more miRNAs in the test sample is:
a) below or substantially below the level of the same one or more miRNAs in the positive control samples; and/or
b) below a predetermined lower threshold value or range of the one or more miRNAs in the positive control samples.

12. The method according to any one or more of paragraphs 1-11 wherein one or more of the miRNAs that represents a miRNA signature indicative of the presence of said pathologically significant intestinal infection or pathologically significant intestinal infection that requires treatment targets one or more genes selected from:

### The Mucin Type O-Glycan Biosynthesis Pathway

13. The method according to any of paragraphs 1-12 wherein one or more of the miRNAs that represent a miRNA signature indicative of the presence of said pathologically significant intestinal infection or pathologically significant intestinal infection that requires treatment targets are selected from:
GALNT16, GALNT6, GALNT12, GALNT14, GALNT5 and GCNT4

14. The method according to any of paragraphs 1-13 wherein the method comprises determining the level of any 1, 2, 3, 4, 5, 6, 7, 8, 9, 10, 11, 12, 13, 14, 15, 16, 17, 18, or 19, or all of the following miRNAs:

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |
| [SEQ ID NO: 9] | gga-let-7c-5p* | TGAGGTAGTAGGTTGTATAGTTT |
| [SEQ ID NO: 10] | gga-miR-142-5p | CCCATAAAGTAGAAAGCACTAC |
| [SEQ ID NO: 11] | gga-miR-23b-3p* | ATCACATTGCCAGGGATTTCC |
| [SEQ ID NO: 12] | gga-miR-7b* | TGGAAGACTAGTGATTTTTGTTGT |
| [SEQ ID NO: 13] | gga-miR-30b-5p | TGTAAACATCCTACACTCAGCT |
| [SEQ ID NO: 14] | gga-miR-219b | CACAAGAATTGCGTTTGGACAA |
| [SEQ ID NO: 15] | tgu-miR-363-3p | AATTGCACGGTATCCATCTGT |
| [SEQ ID NO: 16] | gga-miR-128-3p | TCACAGTGAACCGGTCTCTTT |
| [SEQ ID NO: 17] | gga-miR-19b-3p* | TGTGCAAATCCATGCAAAACTGT |
| [SEQ ID NO: 18] | gga-miR-101-3p* | GTACAGTACTGTGATAACTGA |
| [SEQ ID NO: 19] | gga-miR-183* | TATGGCACTGGTAGAATTCACT |

15. The method according to any of paragraphs 1-14 wherein the method comprises determining the level of any 1, 2, 3, 4, 5, 6, 7 or 8 of the following miRNAs:

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

or of miRNA with a sequence that has at least 90%, 91%, 92%, 93%, 94%, 95%, 96%, 97%, 98%, 99% or 100% sequence identity to the one or more miRNAs of SEQ ID NO: 1-8.

16. The method according to any of paragraphs 1-15 wherein the method comprises determining the level of any 1, 2, 3, 4, or 5 of the following miRNAs:

| [SEQ ID NO: 2] | |
|---|---|
| gga-miR-2188-5p | AAGGTCCAACCTCACATGTCCT |
| [SEQ ID NO: 4] | |
| gga-miR-146c-5p* | TGAGAACTGAATTCCATGGACT |
| [SEQ ID NO: 5] | |
| gga-miR-19a-3p* | TGTGCAAATCCATGCAAAACTGA |
| [SEQ ID NO: 7] | |
| gga-miR-22-3p | AAGCTGCCAGTTGAAGAACTGT |
| [SEQ ID NO: 8] | |
| gga-miR-7* | TGGAAGACTAGTGATTTTGTTGTT |

or of miRNA with a sequence that has at least 90%, 91%, 92%, 93%, 94%, 95%, 96%, 97%, 98%, 99% or 100% sequence identity to the one or more miRNAs of SEQ ID NO: 2, 4, 5, 7 or 8.

17. The method according to any of paragraphs 1-16 wherein the method comprises determining the level of any 1, 2 or 3 of the following miRNAs:

| [SEQ ID NO: 2] | |
|---|---|
| gga-miR-2188-5p | AAGGTCCAACCTCACATGTCCT |
| [SEQ ID NO: 5] | |
| gga-miR-19a-3p* | TGTGCAAATCCATGCAAAACTGA |
| [SEQ ID NO: 7] | |
| gga-miR-22-3p | AAGCTGCCAGTTGAAGAACTGT |

or of miRNA with a sequence that has at least 90%, 91%, 92%, 93%, 94%, 95%, 96%, 97%, 98%, 99% or 100% sequence identity to the one or more miRNAs of SEQ ID NO: 2, 5 or 7.

18. The method according to any one of paragraphs 14-17 wherein the method comprises determining the level of:
a) any 1, 2, 3, 4, 5, 6, 7, 8, 9, 10, 11, 12, 13, 14, 15, 16, 17, 18, or 19, or all of the following miRNAs:

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |
| [SEQ ID NO: 9] | gga-let-7c-5p* | TGAGGTAGTAGGTTGTATAGTTT |
| [SEQ ID NO: 10] | gga-miR-142-5p | CCCATAAAGTAGAAAGCACTAC |
| [SEQ ID NO: 11] | gga-miR-23b-3p* | ATCACATTGCCAGGGATTTCC |
| [SEQ ID NO: 12] | gga-miR-7b* | TGGAAGACTAGTGATTTTTGTTGT |
| [SEQ ID NO: 13] | gga-miR-30b-5p | TGTAAACATCCTACACTCAGCT |
| [SEQ ID NO: 14] | gga-miR-219b | CACAAGAATTGCGTTTGGACAA |
| [SEQ ID NO: 15] | tgu-miR-363-3p | AATTGCACGGTATCCATCTGT |
| [SEQ ID NO: 16] | gga-miR-128-3p | TCACAGTGAACCGGTCTCTTT |
| [SEQ ID NO: 17] | gga-miR-19b-3p* | TGTGCAAATCCATGCAAAACTGT |
| [SEQ ID NO: 18] | gga-miR-101-3p* | GTACAGTACTGTGATAACTGA |
| [SEQ ID NO: 19] | gga-miR-183* | TATGGCACTGGTAGAATTCACT |

b) any 1, 2, 3, 4, 5, 6, 7 or 8 of the following miRNAs:

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

c) any 1, 2, 3, 4, or 5 of the following miRNAs:

| | | |
|---|---|---|
| [SEQ ID NO: 2] | gga-miR-2188-5p | AAGGTCCAACCTCACATGTCCT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

d) any 1, 2 or 3 of the following miRNAs:

| | | |
|---|---|---|
| [SEQ ID NO: 2] | gga-miR-2188-5p | AAGGTCCAACCTCACATGTCCT |
| [SEQ ID NO: 5] | gga-miR-19a-3p* | TGTGCAAATCCATGCAAAACTGA |
| [SEQ ID NO: 7] | gga-miR-22-3p | AAGCTGCCAGTTGAAGAACTGT. |

19. The method according to any of paragraphs 1-18 wherein said level of the one or more miRNAs is determined using reverse transcription followed by qPCR.

20. The method according to any of paragraphs 19 wherein the following primers are used to determine the level of said miRNA:
Where the miRNA is [SEQ ID NO: 1] gga-miR-2188-5p, the forward primer is AAGGTCCAACCTCACATGTCCT [SEQ ID NO: 20] and the reverse primer is the Universal mRQ 3′ Primer supplied Mir-X™ miRNA First-Strand Synthesis and SYBR® qRT-PCR kit;
Where the miRNA is [SEQ ID NO: 2] gga19a-3p*, the forward primer is TGTGCAAATCCATGCAAAACTGA [SEQ ID NO: 21] and the reverse primer is the Universal mRQ 3′ Primer supplied Mir-X™ miRNA First-Strand Synthesis and SYBR® qRT-PCR kit;
Where the miRNA is [SEQ ID NO: 3] gga-miR-22-3p, the forward primer is AAGCTGCCAGTTGAAGAACTGT [SEQ ID NO: 22] and the reverse primer is the Universal mRQ 3′ Primer supplied Mir-X™ miRNA First-Strand Synthesis and SYBR® qRT-PCR kit;
Where the miRNA is [SEQ ID NO: 4] gga7*, the forward primer is TGGAAGACTAGTGATTTTGTTGTT [SEQ ID NO: 23] and the reverse primer is the Universal mRQ 3′ Primer supplied Mir-X™ miRNA First-Strand Synthesis and SYBR® qRT-PCR kit;
Where the miRNA is [SEQ ID NO: 5] gga146c-5p*, the forward primer is TGAGAACTGAATTCCATGGACT [SEQ ID NO: 24] and the reverse primer is the Universal mRQ 3′ Primer supplied Mir-X™ miRNA First-Strand Synthesis and SYBR® qRT-PCR kit;
Where the miRNA is [SEQ ID NO: 6] gga193b-3p*, the forward primer is AACTGGCCCACAAAGTCCCGCT [SEQ ID NO: 25] and the reverse primer is the Universal mRQ 3′ Primer supplied Mir-X™ miRNA First-Strand Synthesis and SYBR® qRT-PCR kit;
Where the miRNA is [SEQ ID NO: 7] gga140-3p*, the forward primer is ACCACAGGGTAGAACCACGGACT [SEQ ID NO: 26] and the reverse primer is the Universal mRQ 3′ Primer supplied Mir-X™ miRNA First-Strand Synthesis and SYBR® qRT-PCR kit; and/or
Where the miRNA is [SEQ ID NO: 8] tgu425-5p*, the forward primer is AATGACACGATCACTCCCGCTGAGC [SEQ ID NO: 27] and the reverse primer is the Universal mRQ 3′ Primer supplied Mir-X™ miRNA First-Strand Synthesis and SYBR® qRT-PCR kit.

21. The method according to any of paragraphs 1-20 wherein the method comprises hybridisation of miRNA to a detection probe.

22. A method for determining a miRNA signature predictive of intestinal infection with an infectious agent in birds, optionally predictive of pathologically significant intestinal infection, or pathologically significant intestinal infection that requires treatment, wherein the method comprises:
a) providing at least:
   a first plurality of faecal and/or caecal samples; and
   a second plurality of faecal and/or caecal samples
wherein each of the faecal and/or caecal samples in the first plurality of faecal and/or caecal samples is taken from a different individual bird from a first population of birds that display a first infection phenotype and wherein each of the faecal and/or caecal samples in the second plurality of faecal and/or caecal samples is taken from a different individual bird from a second population of birds that display a second infection phenotype;
b) determining the relative amounts of a range of miRNAs in the first plurality of faecal and/or caecal samples and in the second plurality of faecal and/or caecal samples;
c) identifying miRNAs that are present in statistically different amounts between the first plurality of faecal and/or caecal samples level and the second plurality of faecal and/or caecal samples, and thereby identifying the miRNA signature predictive of infection with the infectious agent, optionally predictive of pathologically significant intestinal infection or pathologically significant intestinal infection that requires treatment.

23. The method according to paragraph 22 wherein in step (a) a third plurality of faecal and/or caecal samples that has a third infection phenotype is provided.

24. The method according to paragraph 22 or 23 wherein the first infection phenotype is “uninfected” or “no pathologically significant level of infection” or “no pathologically significant level of infection that requires treatment” and the second infection phenotype is “infected” or “pathologically significant infection” or “pathologically significant level of infection that requires treatment”.

25. The method according to paragraph 22-24 wherein:
where the first infection phenotype is “uninfected”; a the second infection phenotype is “low level of infection”; and the third phenotype is “high level of infection”.

26. The method according to any of paragraphs 22-25 wherein the infectious agent is a parasite, optionally is:
a) Eimeralla sp;
b) selected from Eimeria Tenella, Eimeria necatrix, Eimeria acervuline, Eimeria brunetti, Eumeria maxima, Eimeria mitis, Eimeria praecox;
c) is Eimeria tenella or Eimeria necatrix; or
d) Eimeria tenella.

27. The method according to any of paragraphs 22-25 wherein the infectious agent is a bacteria, optionally is Salmonella sp or E. coli.

28. A kit comprising one or more or all of the following:
a) Means to determine the level of at least one of the miRNAs associated with the presence of said avian intestinal parasite;
b) means for the extraction and purification of RNA from a caecal and/or faecal sample;
c) a control sample of RNA prepared from a faecal sample from subjects that do not comprise the intestinal parasite.

29. The kit according to paragraph 28 wherein the means to determine the level of at least one of the miRNAs comprises:
A) at least one pair of primers are arranged so as to amplify at least one or more of the following miRNAs:

i)

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |
| [SEQ ID NO: 9] | gga-let-7c-5p* | TGAGGTAGTAGGTTGTATAGTTT |
| [SEQ ID NO: 10] | gga-miR-142-5p | CCCATAAAGTAGAAAGCACTAC |
| [SEQ ID NO: 11] | gga-miR-23b-3p* | ATCACATTGCCAGGGATTTCC |
| [SEQ ID NO: 12] | gga-miR-7b* | TGGAAGACTAGTGATTTTTGTTGT |
| [SEQ ID NO: 13] | gga-miR-30b-5p | TGTAAACATCCTACACTCAGCT |
| [SEQ ID NO: 14] | gga-miR-219b | CACAAGAATTGCGTTTGGACAA |
| [SEQ ID NO: 15] | tgu-miR-363-3p | AATTGCACGGTATCCATCTGT |
| [SEQ ID NO: 16] | gga-miR-128-3p | TCACAGTGAACCGGTCTCTTT |
| [SEQ ID NO: 17] | gga-miR-19b-3p* | TGTGCAAATCCATGCAAAACTGT |
| [SEQ ID NO: 18] | gga-miR-101-3p* | GTACAGTACTGTGATAACTGA |
| [SEQ ID NO: 19] | gga-miR-183* | TATGGCACTGGTAGAATTCACT |

ii)

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

iii)

| | | |
|---|---|---|
| [SEQ ID NO: 2] | gga-miR-2188-5p | AAGGTCCAACCTCACATGTCCT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

or iv)

| | | |
|---|---|---|
| [SEQ ID NO: 2] | gga-miR-2188-5p | AAGGTCCAACCTCACATGTCCT |
| [SEQ ID NO: 5] | gga-miR-19a-3p* | TGTGCAAATCCATGCAAAACTGA |
| [SEQ ID NO: 7] | gga-miR-22-3p | AAGCTGCCAGTTGAAGAACTGT. |

and/or
B) one or more hybridisation probes able to specifically hybridise to one or more of the following miRNAs:

i)

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |
| [SEQ ID NO: 9] | gga-let-7c-5p* | TGAGGTAGTAGGTTGTATAGTTT |
| [SEQ ID NO: 10] | gga-miR-142-5p | CCCATAAAGTAGAAAGCACTAC |
| [SEQ ID NO: 11] | gga-miR-23b-3p* | ATCACATTGCCAGGGATTTCC |
| [SEQ ID NO: 12] | gga-miR-7b* | TGGAAGACTAGTGATTTTTGTTGT |
| [SEQ ID NO: 13] | gga-miR-30b-5p | TGTAAACATCCTACACTCAGCT |
| [SEQ ID NO: 14] | gga-miR-219b | CACAAGAATTGCGTTTGGACAA |
| [SEQ ID NO: 15] | tgu-miR-363-3p | AATTGCACGGTATCCATCTGT |
| [SEQ ID NO: 16] | gga-miR-128-3p | TCACAGTGAACCGGTCTCTTT |
| [SEQ ID NO: 17] | gga-miR-19b-3p* | TGTGCAAATCCATGCAAAACTGT |
| [SEQ ID NO: 18] | gga-miR-101-3p* | GTACAGTACTGTGATAACTGA |
| [SEQ ID NO: 19] | gga-miR-183* | TATGGCACTGGTAGAATTCACT |

ii)

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

iii)

| | | |
|---|---|---|
| [SEQ ID NO: 2] | gga-miR-2188-5p | AAGGTCCAACCTCACATGTCCT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

or iv)

| | | |
|---|---|---|
| [SEQ ID NO: 2] | gga-miR-2188-5p | AAGGTCCAACCTCACATGTCCT |
| [SEQ ID NO: 5] | gga-miR-19a-3p* | TGTGCAAATCCATGCAAAACTGA |
| [SEQ ID NO: 7] | gga-miR-22-3p | AAGCTGCCAGTTGAAGAACTGT. |

30. A kit comprising:
A) any one or more of the following primers:

Forward primer
[SEQ ID NO: 20]
AAGGTCCAACCTCACATGTCCT
Forward primer
[SEQ ID NO: 21]
TGTGCAAATCCATGCAAAACTGA
Forward primer
[SEQ ID NO: 22]
AAGCTGCCAGTTGAAGAACTGT
Forward primer
[SEQ ID NO: 23]
TGGAAGACTAGTGATTTTGTTGTT
Forward primer
[SEQ ID NO: 24]
TGAGAACTGAATTCCATGGACT
Forward primer
[SEQ ID NO: 25]
AACTGGCCCACAAAGTCCCGCT
Forward primer
[SEQ ID NO: 26]
ACCACAGGGTAGAACCACGGACT
Forward primer
[SEQ ID NO: 27]
AATGACACGATCACTCCCGCTGAGC;

And comprising
B) a suitable reverse primer, optionally a reverse primer that is the Universal mRQ 3′ Primer supplied Mir-X™ miRNA First-Strand Synthesis and SYBR® qRT-PCR kit.

31. The kit according to paragraph 30 comprising:

A)
Forward primer
[SEQ ID NO: 21]
AAGCTGCCAGTTGAAGAACTGT;
Forward primer
[SEQ ID NO: 24]
TGAGAACTGAATTCCATGGACT;
and
Forward primer
[SEQ ID NO: 26]
ACCACAGGGTAGAACCACGGACT;

and
B) a suitable reverse primer, optionally a reverse primer that is the Universal mRQ 3′ Primer supplied Mir-X™ miRNA First-Strand Synthesis and SYBR® qRT-PCR kit.

32. The kit according to paragraph 30 or 31 comprising:

A)
Forward primer
[SEQ ID NO: 21]
AAGCTGCCAGTTGAAGAACTGT;
Forward primer
[SEQ ID NO: 24]
TGAGAACTGAATTCCATGGACT;
Forward primer
[SEQ ID NO: 26]
ACCACAGGGTAGAACCACGGACT;
Forward primer
[SEQ ID NO: 23]
TGGAAGACTAGTGATTTTGTTGTT;
and
Forward primer
[SEQ ID NO: 27]
AATGACACGATCACTCCCGCTGAGC;

and
B) a suitable reverse primer, optionally a reverse primer that is the Universal mRQ 3′ Primer supplied Mir-X™ miRNA First-Strand Synthesis and SYBR® qRT-PCR kit.

33. The kit according to any of paragraphs 30-32 comprising:

(A)
Forward primer
[SEQ ID NO: 20]
AAGGTCCAACCTCACATGTCCT;
Forward primer
[SEQ ID NO: 21]
TGTGCAAATCCATGCAAAACTGA;
Forward primer
[SEQ ID NO: 22]
AAGCTGCCAGTTGAAGAACTGT;
Forward primer
[SEQ ID NO: 23]
TGGAAGACTAGTGATTTTGTTGTT;
Forward primer
[SEQ ID NO: 24]
TGAGAACTGAATTCCATGGACT;
Forward primer
[SEQ ID NO: 25]
AACTGGCCCACAAAGTCCCGCT;
Forward primer
[SEQ ID NO: 26]
ACCACAGGGTAGAACCACGGACT;
and
Forward primer
[SEQ ID NO: 27]
AATGACACGATCACTCCCGCTGAGC;

and
B) a suitable reverse primer, optionally a reverse primer that is the Universal mRQ 3′ Primer supplied Mir-X™ miRNA First-Strand Synthesis and SYBR® qRT-PCR kit.

34. The method according to any of paragraphs 1-27 wherein the determination of the presence of an infection with an infectious agent, optionally presence of pathologically significant intestinal infection or pathologically significant intestinal infection that requires treatment indicates that the subject has a high lesion score (severe lesions), optionally a lesion score of 4.

35. The method according to any of paragraphs 1-27 and 34 wherein upon the determination of the presence of infection with an infectious agent, optionally determination of pathologically significant intestinal infection or pathologically significant intestinal infection that requires treatment, the subject is treated with a therapeutic to treat infection with the avian intestinal infection.

36. The method according to any of paragraphs 1-27, 34 and 35 wherein upon determination of the presence of infection with an infectious agent, optionally determination of pathologically significant intestinal infection or pathologically significant intestinal infection that requires treatment, the subject or subjects are isolated from other flocks of birds.

37. A method for determining the efficacy of a treatment against one or more intestinal infections in a bird wherein the method comprises:
a) determining the level of one or more miRNAs in a sample of the faeces and/or caecal content from the one or more test birds, wherein the one or more miRNAs represents a miRNA signature indicative of the presence of said pathologically significant intestinal infection or pathologically significant intestinal infection that requires treatment according to the method of any of paragraphs 1-27 and 34-36;
b) confirming the presence of said pathologically significant intestinal infection or pathologically significant intestinal infection that requires treatment based upon the level of the one or more miRNAs determined in (a);
c) treating said bird with an appropriate therapeutic to treat said intestinal infection;
d) following the treatment in step (c), repeating step (a);
e) confirming the presence of said pathologically significant intestinal infection or pathologically significant intestinal infection that requires treatment based upon the level of the one or more miRNAs determined in (d).

38. A method for determining the efficacy of a virulent vaccine against intestinal infection in a bird wherein the method comprises determining the presence of said intestinal infection, or pathologically significant intestinal infection or pathologically significant intestinal infection that requires treatment according to the method of any one or more of paragraphs 1-27 and 34-36 following administration of the vaccination.

39. The method according to paragraph 38 wherein the virulent vaccine is considered to have been effective if the presence of the avian intestinal parasite is detected.

40. A method for detecting harmful effects of a virulent vaccine against intestinal infection in a bird wherein the method comprises:
a) determining the presence of said intestinal infection, or pathologically significant intestinal infection or pathologically significant intestinal infection that requires treatment according to the method of any one or more of paragraphs 1-27 and 34-36 following administration of the vaccination at:
   i) a first time point; and
   ii) a second time point;
wherein the first time point and second time point are selected so as to allow determination of persistent presence of the intestinal infection, wherein persistent presence of the intestinal infection indicates harmful effects of the virulent vaccine.

41. A method for screening a population of birds the presence of an avian intestinal parasite wherein the method comprises determining the presence of an avian intestinal parasite according to any of the preceding paragraphs wherein the sample is a sample of faecal matter taken from the avian environment.

42. The method according to paragraph 41 wherein the sample comprises a fecal matter from a number of individual birds from the avian environment.

43. The method according any of paragraphs 41 or 42 wherein the method for screening is performed at periodic intervals, optionally at intervals of 1 week, 2 weeks, 3 weeks, 4 weeks, 1 month, 2 months, 3 months, 4 months, 5 months or 6 months or more.

44. The method according to paragraph 43 wherein the method comprises comparing the level of the one or more miRNAs in the sample between intervals to determine an elevation in the level of the one or more miRNAs, wherein an increase in the level of the one or more miRNAs indicates an increased presence of an avian intestinal parasite in the population of chickens.

45. The method according to paragraph 44 wherein once an increase in the level of the one or more miRNAs is detected, the population is treated with a therapeutic agent to treat the avian intestinal infection.

46. The method according to any preceding embodiment wherein:
where the expression level of [SEQ ID NO: 1] gga-miR-193b-3p*AACTGGCCCACAAAGTCCCGCT is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 2] gga-miR-2188-5p AAGGTCCAACCTCACATGTCCT is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 3] gga-miR-140-3p*ACCACAGGGTAGAACCACGGACT is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 4] gga-miR-146c-5p*TGAGAACTGAATTCCATGGACT is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 5] gga-miR-19a-3p*TGTGCAAATCCATGCAAAACTGA is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 6] tgu-miR-425-5p*AATGACACGATCACTCCCGCTGAGC is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 7] gga-miR-22-3p AAGCTGCCAGTTGAAGAACTGT is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 8] gga-miR-7*TGGAAGACTAGTGATTTTGTTGTT is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 9] gga-let-7c-5p*TGAGGTAGTAGGTTGTATAGTTT is down regulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 10] gga-miR-142-5p CCCATAAAGTAGAAAGCACTAC is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 11] gga-miR-23b-3p* ATCACATTGCCAGGGATTTCC is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 12] gga-miR-7b* TGGAAGACTAGTGATTTTTGTTGT is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 13] gga-miR-30b-5p TGTAAACATCCTACACTCAGCT is down regulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 14] gga-miR-219b CACAAGAATTGCGTTTGGACAA is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 15] tgu-miR-363-3p AATTGCACGGTATCCATCTGT is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 16] gga-miR-128-3p TCACAGTGAACCGGTCTCTTT is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 17] gga-miR-19b-3p* TGTGCAAATCCATGCAAAACTGT is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 18] gga-miR-101-3p* GTACAGTACTGTGATAACTGA is upregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;
where the expression level of [SEQ ID NO: 19] gga-miR-183* TATGGCACTGGTAGAATTCACT is downregulated with respect to the expression level of the miRNA in a negative control, the test bird is considered to have an infection with the infectious agent, optionally with Eimeria sp, for example Eimeria tenella, or is considered to have a pathologically significant with the infectious agent, optionally with Eimeria sp, for example Eimeria tenella;

| | | | Up or |
|---|---|---|---|
| SEQ ID | miRbase ID | Sequence | down |
| [SEQ ID NO: 1] | gga-miR-193b-3p* | AACTGGCCCACAAAGTCCCGCT | Up |
| [SEQ ID NO: 2] | gga-miR-2188-5p | AAGGTCCAACCTCACATGTCCT | Up |
| [SEQ ID NO: 3] | gga-miR-140-3p* | ACCACAGGGTAGAACCACGGACT | Up |
| [SEQ ID NO: 4] | gga-miR-146c-5p* | TGAGAACTGAATTCCATGGACT | Up |
| [SEQ ID NO: 5] | gga-miR-19a-3p* | TGTGCAAATCCATGCAAAACTGA | Up |
| [SEQ ID NO: 6] | tgu-miR-425-5p* | AATGACACGATCACTCCCGCTGAGC | Up |
| [SEQ ID NO: 7] | gga-miR-22-3p | AAGCTGCCAGTTGAAGAACTGT | Up |
| [SEQ ID NO: 8] | gga-miR-7* | TGGAAGACTAGTGATTTTGTTGTT | Up |
| [SEQ ID NO: 9] | gga-let-7c-5p* | TGAGGTAGTAGGTTGTATAGTTT | Down |
| [SEQ ID NO: 10] | gga-miR-142-5p | CCCATAAAGTAGAAAGCACTAC | Up |
| [SEQ ID NO: 11] | gga-miR-23b-3p* | ATCACATTGCCAGGGATTTCC | Up |
| [SEQ ID NO: 12] | gga-miR-7b* | TGGAAGACTAGTGATTTTTGTTGT | |
| [SEQ ID NO: 13] | gga-miR-30b-5p | TGTAAACATCCTACACTCAGCT | Down |
| [SEQ ID NO: 14] | gga-miR-219b | CACAAGAATTGCGTTTGGACAA | Up |
| [SEQ ID NO: 15] | tgu-miR-363-3p | AATTGCACGGTATCCATCTGT | Up |
| [SEQ ID NO: 16] | gga-miR-128-3p | TCACAGTGAACCGGTCTCTTT | Up |
| [SEQ ID NO: 17] | gga-miR-19b-3p* | TGTGCAAATCCATGCAAAACTGT | Up |
| [SEQ ID NO: 18] | gga-miR-101-3p* | GTACAGTACTGTGATAACTGA | Up |
| [SEQ ID NO: 19] | gga-miR-183* | TATGGCACTGGTAGAATTCACT | Down |

### REFERENCES

Johnson, J. & Reid, W. M. (1970). Anticoccidial drugs: lesion scoring techniques in battery and floor-pen experiments with chickens. Experimental Parasitology, 28, 30-36.
Liu, T.-L., Fan, X.-C., Wang, Y., Wang, Y.-X., Wang, J.-W., Song, J.-K. & Zhao, G.-H. (2020). Micro-RNA expression profile of chicken small intestines during Eimeria necatrix infection. Poultry Science, 99, 2444-2451.
Liu, X., Liu, L., Zhang, M., Wang, H., Yang, N. & Li, X. (2016). Chicken cecal microRNAs in the response to Campylobacter jejuni inoculation by Solexa sequencing. Poultry Science, 95, 2819-2823.
Livak, K. J. & Schmittgen, T. D. (2001). Analysis of relative gene expression data using real-time quantitative PCR and the 2(-Delta Delta C (T)) Method. Methods (San Diego, Calif.), 25, 402-408.
Long, P. L., Millard, B. J., Joyner, L. P. & Norton, C. C. (1976). A guide to laboratory techniques used in the study and diagnosis of avian coccidiosis. Folia Veterinaria Latina, 6, 201-217.
Bergstrom, K. S., and L. Xia
   2013 Mucin-type O-glycans and their roles in intestinal homeostasis. Glycobiology 23 (9): 1026-37.
Blake, D. P., et al.
   2020 Re-calculating the cost of coccidiosis in chickens. Veterinary Research 51:115.
Chapman, H. D.
   1999 Anticoccidial drugs and their effects upon the development of immunity to Eimeria infections in poultry. Avian Pathol 28 (6): 521-35.
Chapman, H. D., et al.
   2013 A selective review of advances in coccidiosis research. Adv Parasitol 83:93-171.
Chapman, H. D., and T. K. Jeffers
   2014 Vaccination of chickens against coccidiosis ameliorates drug resistance in commercial poultry production. Int. J Parasitol Drugs Drug Resist 4 (3): 214-7.
Elwinger, K, et al.
   2016 A brief history of poultry nutrition over the last hundred years. World's Poultry Science Journal 72:701-720.
Macdonald, S. E., et al.
   2017 Effects of Eimeria tenella infection on chicken caecal microbiome diversity, exploring variation associated with severity of pathology. PLOS One 12 (9): e0184890.
Peek, H. W., and W. J. Landman
   2003 Resistance to anticoccidial drugs of Dutch avian Eimeria spp. field isolates originating from 1996, 1999 and 2001. Avian Pathol 32 (4): 391-401.
Tierney, J. B., et al.
   2007 INTERACTION OF EIMERIA TENELLA WITH INTESTINAL MUCIN IN VITRO. Journal of Parasitology 93 (3): 634-638.
Xuan, Lijia, et al.
   2019 MicroRNAs regulating mucin type O-glycan biosynthesis and transforming growth factor B signaling pathways in nasal mucosa of patients with chronic rhinosinusitis with nasal polyps in Northern China. International Forum of Allergy & Rhinology 9 (1): 106-113.

### Figure Legends

FIG. 1: Box- and-whisker plots of spectrophotometry and bioanalyser assessment of caecal content extracted by Norgen and Mirvana kits. RNA concentration in Norgen and Mirvana extraction kits (A). RIN values for each extraction method in all samples (B). RNA concentration in the uninfected population for each extraction kit (C). RNA concentration in the infected population for each extraction kit (D). Whiskers represent minimum and maximum, box represents 25th and 75th centiles and line represents median.

FIG. 2: Box- and-whisker plots of spectrophotometry assessment of caecal content extracted by Norgen and Mirvana kits. 260/280 ratio in Norgen and Mirvana extraction kits (A). 260/230 ratio in Norgen and Mirvana extraction kits (B). Whiskers represent minimum and maximum, box represents 25th and 75th centiles and line represents median.

FIG. 3: A-Heatmap showing hierarchical clustering of differentially expressed MiRNAs between control (X1-3) low lesion score (X4-6) and high lesion score (X7-9) in sequencing of caecal content. B-expanded portion of the lower part of the heatmap of part A.

FIG. 4: Box plot of significantly differentially expressed MiRNAs between control, low lesion score and high lesion score in sequencing of caecal content. Whiskers represent minimum and maximum, box represents 25th and 75th centiles and line represents median.

FIG. 5: qPCR of differentially expressed miRNAs performed on caecal contents of biological replicates identified from sequencing data in control (C; n=3) Eimeria tenella infected high lesion score (H; n=3) and low lesion score (L; n=3). Error bars represent standard deviation of biological replicates. ***=P<0.0001, **=P<0.01, and *=P<0.05 compared to control. #=P<0.05 and ###=P<0.001 compared to high lesion score. Cel39-3p is a miRNA present in C. elegans and represents a negative control.

FIG. 6: qPCR of differentially expressed miRNAs identified from sequencing data in control (C; n=4) and Eimeria tenella infected high lesions score (H; n=5) faecal content. Error bars represent standard deviation of biological replicates. **=P<0.01 compared to control.

FIG. 7: Sequences of selected miRNAs identified in this study and used as primers for validation qPCR. Note, the reverse primer used in validation for all target miRNAs was the Universal mRQ 3′ Primer supplied with the Mir-X™ miRNA First-Strand Synthesis and SYBR® qRT-PCR kit. cel-miR-39 represents a non-target sequence from C. Elegans used as a negative control.

### Examples

### Example 1 Extraction Methods

To assess our ability to recover miRNAs from intestinal contents we compared the concentration and quality of RNA extracted from chicken caecal content after storage for approximately 16 months at −80° C. using two commercially available RNA extraction kits (Norgen Stool Total RNA Extraction Kit and Mirvana miRNA Isolation Kit). The concentrations and optical densities of the extracted samples were analysed using spectrophotometry. Concentration analysis produced a mean of 131.62 ng/μl+51.74 (SD) using the Norgen kit and a mean of 264.04 ng/μl #147.69 (SD) with the Mirvana kit.

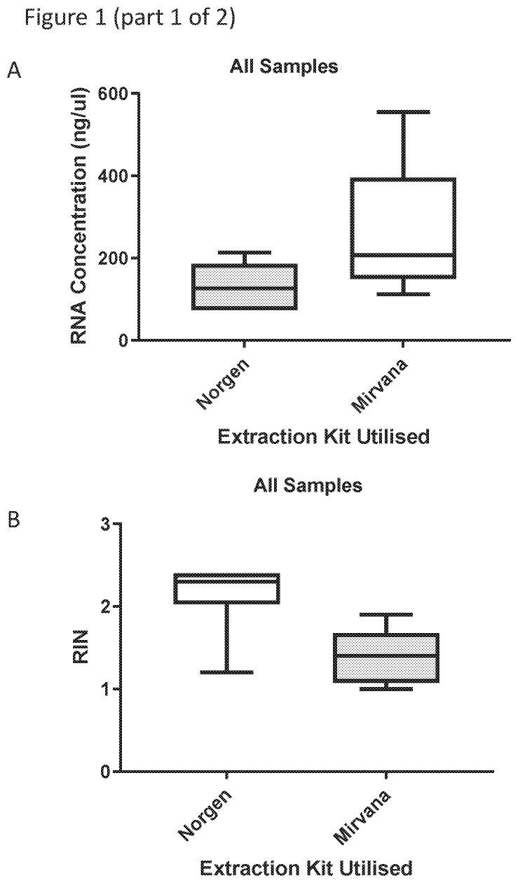

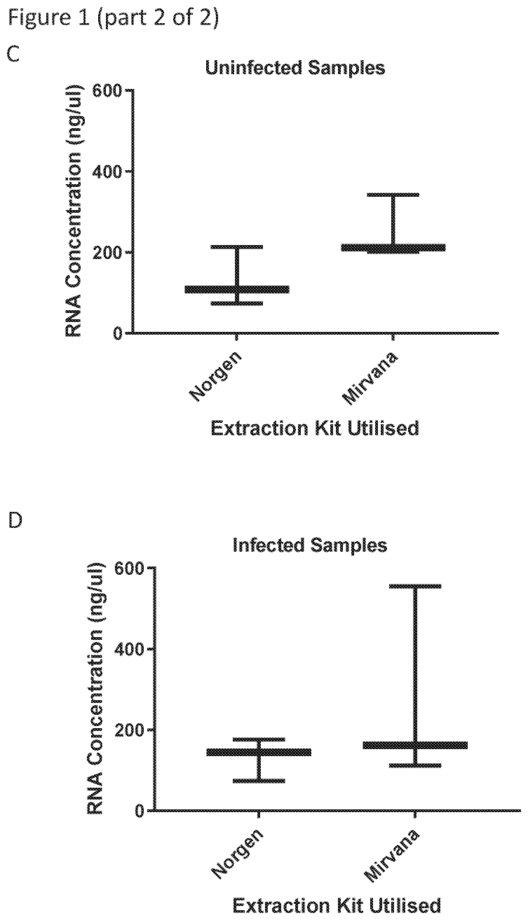

The Mirvana kit demonstrated a wider range of concentrations than the Norgen kit (FIG. 1). Samples extracted by the Mirvana kit yielded a 44% mean increase in concentration compared to the Norgen kit.

Comparing uninfected and infected samples for both kits gave ranges of 73.68-342.35 ng/μl (with a mean of 191.81 ng/μl) and 73.71-554.71 ng/μl (with a mean of 203.84 ng/μl), respectively (FIG. 1). When the Norgen uninfected and infected samples were compared, the uninfected samples gave a range of 73.68-213.08 ng/μl whilst the infected samples gave a range of 73.71-176.39 ng/μl. When the Mirvana uninfected and infected samples were compared, the uninfected samples gave a range of 201.78-342.35 ng/μl whilst the infected samples gave a range of 111.57-554.71 ng/μl (FIG. 1).

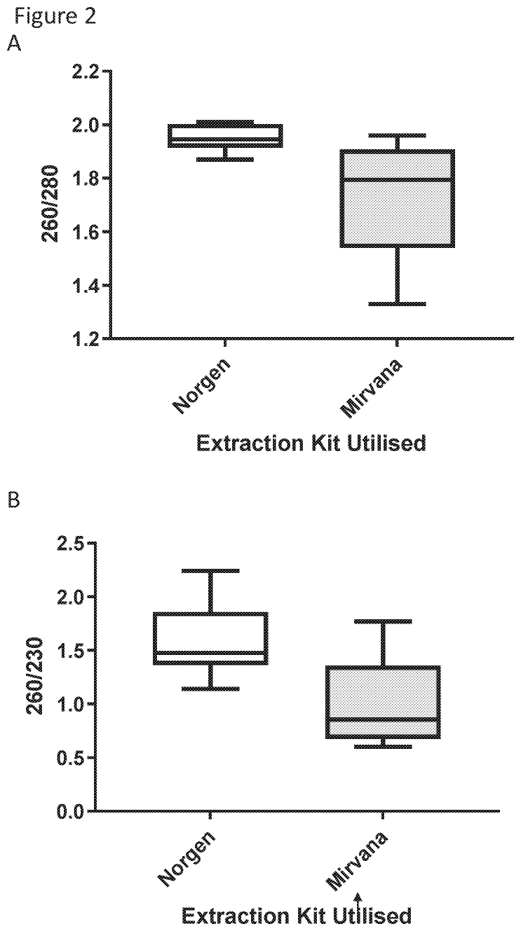

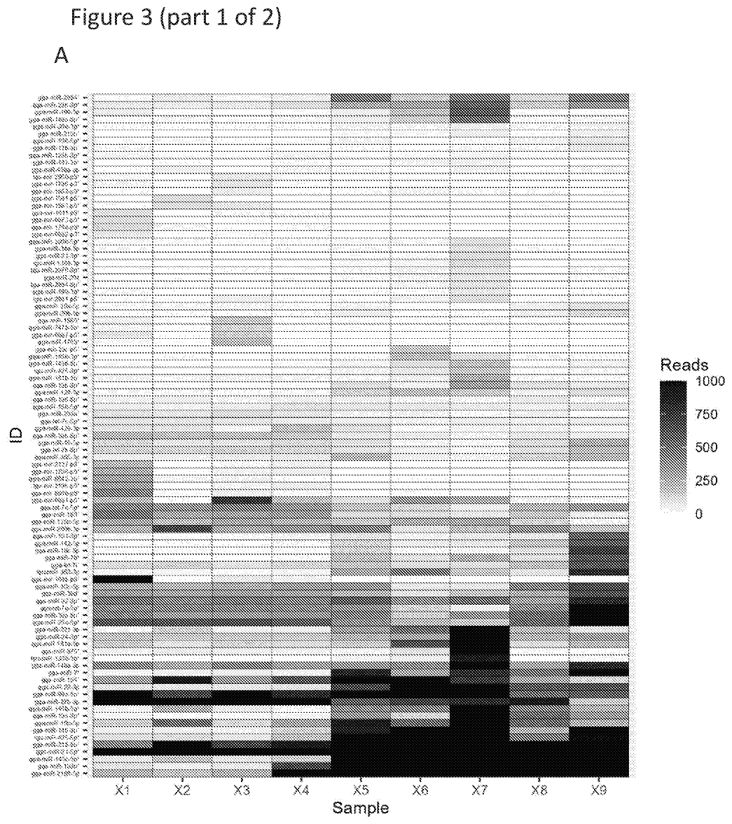

The Norgen kit gave a 260/280 ratio range of 1.87-2.01 and a mean of 1.95, whilst the Mirvana kit gave a range of 1.33-1.96 and a mean of 1.73, suggesting that samples extracted using the Norgen kit resulted in lower levels of protein contamination overall (FIG. 2). The Norgen kit gave a 260/230 ratio range of 1.14-2.24 and an average of 1.58, whilst the Mirvana kit gave a range of 0.6-1.77 and an average of 1.0, inferring that samples extracted using the Norgen kit also resulted in lower levels of organic contamination overall. Both kits produced low RIN values when assessed by Bioanalyzer, as was expected for long term stored caecal content; the Norgen samples gave a range of 1.2-2.4 (mean 2.2) and the Mirvana samples gave a range of 1-1.9 (mean 1.4).

### Example 2 miRNA Sequencing

Following validation of RNA extraction and miRNA amplification from caecal content, we proceeded to sequence miRNA in caecal content samples collected from 26 day old Cobb500 broiler chickens.

Sequencing generated 181,950,730 raw reads, of which 81,013,797 were mappable against pre- and/or mature miRNAs in miRbase following exclusion of adapter and contaminant reads, reads that were <15 or >32 bases in length after removal of the 3′ adapter (3ADT), or where the 3′ adapter was not present. Identified miRNAs represented by 82,755 reads could be mapped to the G. gallus genome, with a further 1,395,452 reads mapped directly to the genome when the associated pre-miRNA identified in miRbase mapped to an avian species other than G. gallus (Table 2, groups 1a and 2a+2b, respectively). A further 57,917 reads were mapped in miRbase to avian mature and/or pre-miRNAs, but did not map to G. gallus miRNAs or genome (Table 1, group 3a). From the remaining reads 9,669,508 did not map to miRbase but could be mapped to the G. gallus genome (16.3%), while 43,867,841 reads had no hit in any of the databases (54.1%). All remaining reads mapped to mRNAs or other RNAs.

Differentially Expressed miRNAS

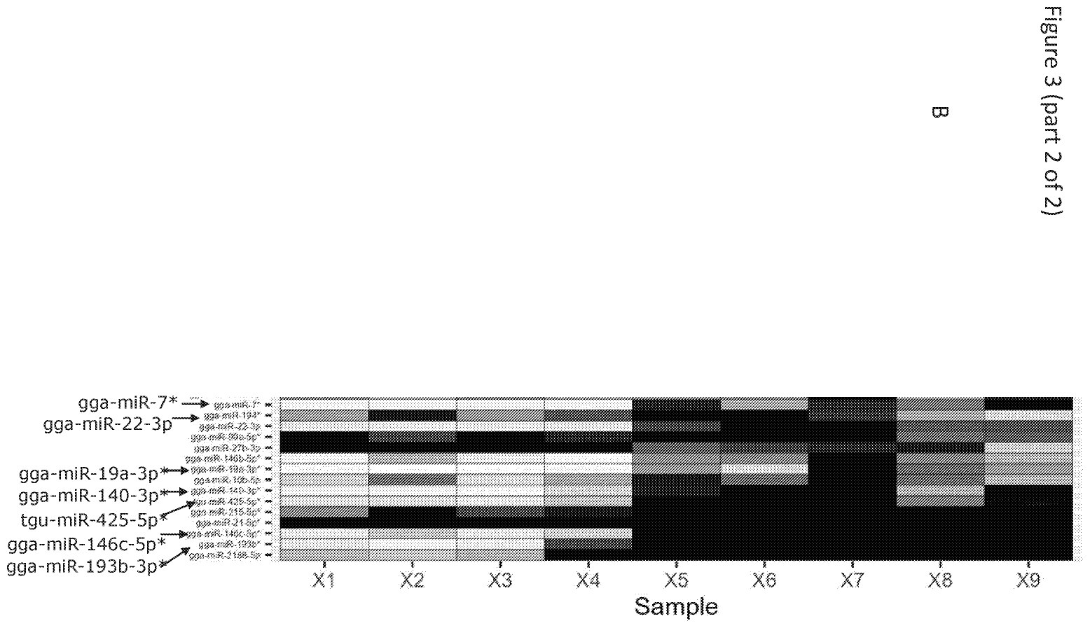

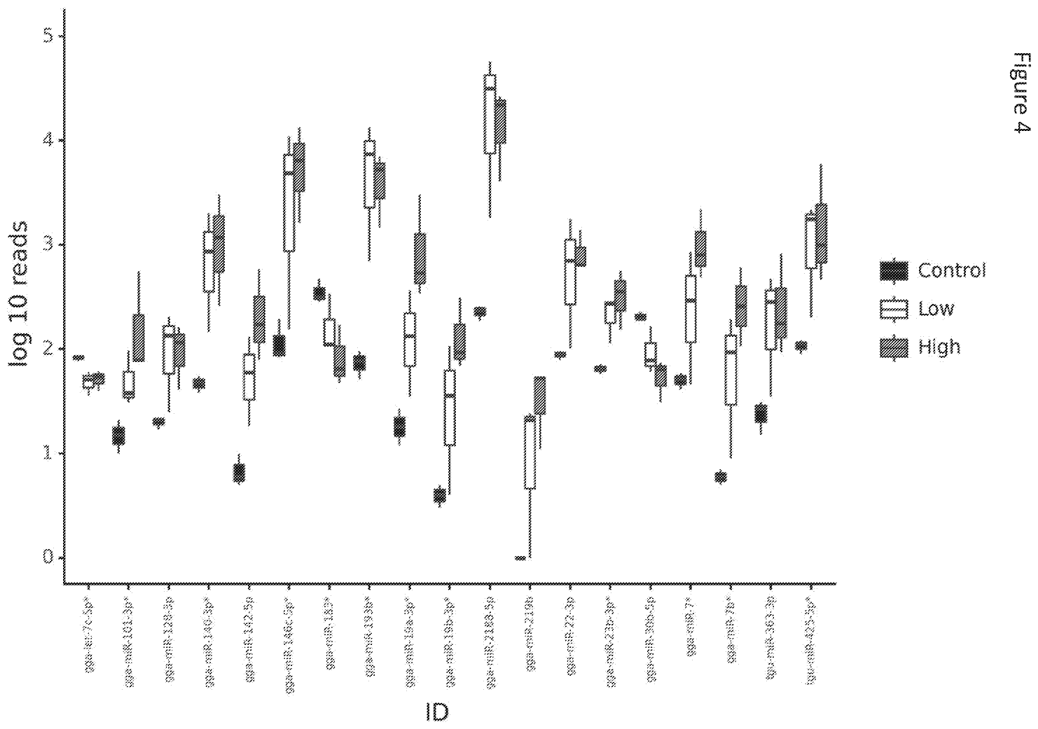

Statistical analysis showed 19 miRNAs to exhibit significantly altered expression in the caecal content of E. tenella infected chickens, irrespective of lesion score, when compared to uninfected controls (This included 16 upregulated miRNAs and 3 down regulated miRNAs (FIG. 4; t-test, False Discovery Rate (FDR)<0.05)). Eight of these miRNAs showing marked up-regulation in infection were selected for further validation, including MiRNA gga-miR-7* (15 fold increase), gga-miR-2188-5p (105 fold increase), gga-miR-193b-3p* (78 fold increase), gga-miR-146c-5p* (51 fold increase), gga-miR-19a-3p* (39 fold increase), gga-miR-140-3p* (26 fold increase) and gga-miR-22-3p (10 fold increase) from group 2a, and MiRNA tgu-miR-425-5p* (18 fold increase). (FIG. 4).

TABLE 1
Read numbers from sequencing of chicken caecal RNA separated by mapping
to known chicken (Gallus domesticus) or other avian miRNAs, the chicken
genome, and the presence of absence of predicted hairpins. The number of
known and predicted unique miRNAs are identified within each group.

| | | | Unique miRNAs |
|---|---|---|---|
| Group description | No. of reads | | identified |
| 1a: Mapped to known mature miRNAs of Gallus | 82,755 | (0.1%) | 64 |
| 2a: Mapped to known avian pre-miRNAs and G. gallus | 1,202,120 | (1.5%) | 287 |
| genome; predicted to form hairpins | | | |
| 2b: Mapped to known avian pre-miRNAs and G. gallus | 193,332 | (0.2%) | 318 |
| genome; not predicted to form hairpins | | | |
| 3a: Mapped to known avian pre- and mature miRNAs, | 57,917 | (0.1%) | 628 |
| but not mapped to G. gallus genome | | | |
| 3b: Mapped to known avian pre-miRNAs but not mapped | 849 | (0.0%) | 130 |
| to G. gallus genome | | | |
| 4a: Mapped to G. gallus genome but not mapped to | 197,761 | (0.2%) | 766 |
| known avian pre-miRNAs, predicted to form hairpins | | | |
| 4b: Mapped to G. gallus genome but not mapped to | 9,471,747 | (11.7%) | N/A |
| known avian pre-miRNAs, not predicted to form hairpins | | | |
| Other (mapped to mRNA, RFam, or repbase) | 25,939,470 | (32.0%) | N/A |
| No hit | 43,867,841 | (54.1%) | N/A |

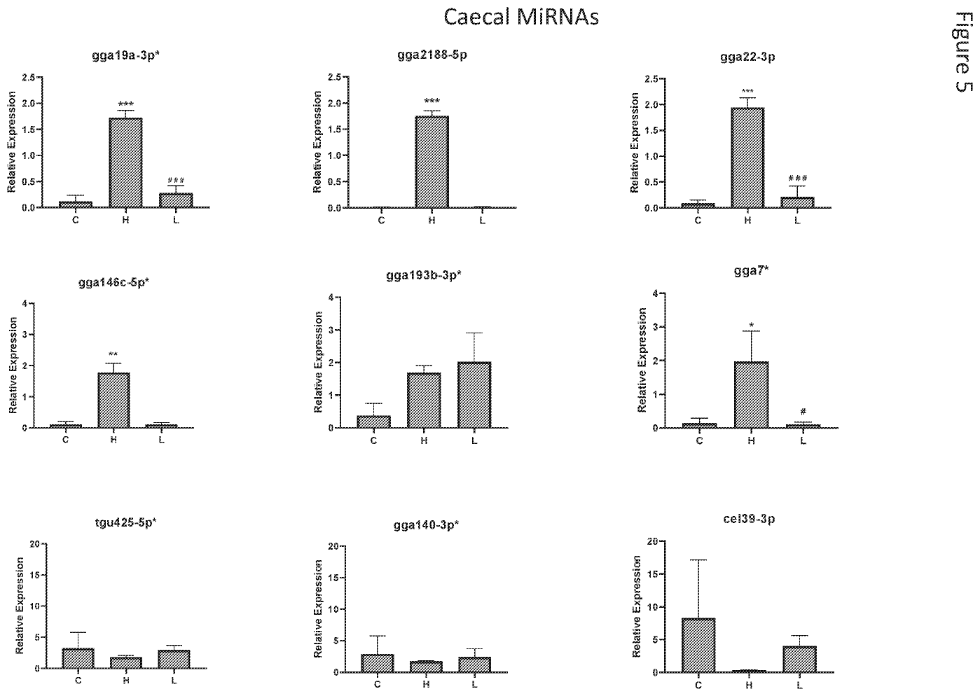

While significantly altered reads were identified in lesion score 4 samples compared to lesion score 1 (FIG. 5; FDR<0.05), only one of these represented a miRNA from Group 2a, whereas 7 of these were of Group 4a and demonstrated low read numbers (less than the mean of the data set).

### Example 4 Functional Interpretation

Following identification of the 19 significantly differentially expressed miRNAs, we queried the miRNet (https://www.mirnet.ca), gene ontology resource (http://geneontology.org/) and Database for Annotation, Visualization and Integrated Discovery (DAVID) (https://david.ncifcrf.gov/) databases to identify predicted gene targets and subsequent downstream proteins, pathways, and biological processes which may be affected. Querying the gene ontology database, we found that predicted targets for these miRNAs included 384 genes. While statistical testing via the gene ontology database and DAVID did not yield statistically significantly altered pathways, hypergeometric testing of the KEGG database via miRNet (miRnada database) showed significant over representation of the Mucin type O-Glycan biosynthesis pathway (adjusted P value of 0.0016), showing 6 hits for gene targets GALNT16, GALNT6, GALNT12, GALNT14, GALNT5, GCNT4. Of these differentially regulated genes, GALNT5 showed co-regulation by more than one miRNA (namely gga-miR-193b and gga-miR-128-3p).

### Example 5 qPCR of miRNA Targets Selected for Validation from the RNAseq Data

In order to test the reproducibility of differentially expressed miRNAs in E. tenella infection, a second set of samples were collected from a duplicate experiment run 20 months later, providing independent biological replication which included caecal content from uninfected controls (n=3), low lesion score 0/1 (n=3) and high lesion score 4 (n=3).

qPCR corroborated significant differential expression of 5 of the 8 upregulated miRNAs identified from sequencing (FIG. 5). This demonstrated significant differential expression in samples from infected chickens with high lesion scores (H) compared to infected birds with low lesion scores (L) and uninfected control birds (C). This included a 292-fold increase of gga2188-5p in the H group (P<0.001, relative expression of 1.75+/−0.01 compared to 0.01+/−0.01 in controls, and 0.01+/−0.01 in the L group), a 16-fold increase of gga146c-5p_R-1 in the H group (relative expression of 1.77+/−0.31 compared to 0.11+/−0.01 in the C group, and 0.11+/−0.06 in the L group), 15-fold increase of gga19a-3p_1ss11TC in H group (relative expression of 1.72+/−0.14 compared to 0.12+/−0.12 in C group, and 0.28+/−0.14 in L group), 22 fold increase in gga22-3p in H group (relative expression of 1.94+/−0.19 compared to 0.09+/−0.06 in C group, and 0.21+/−0.21 in L group) and 14 fold increase in gga7 in H group (relative expression of 1.98+/−0.90 compared to 0.15+/−0.15 in C group, and 0.11+/−0.0.07 in L group).

### Validation in Faecal Samples

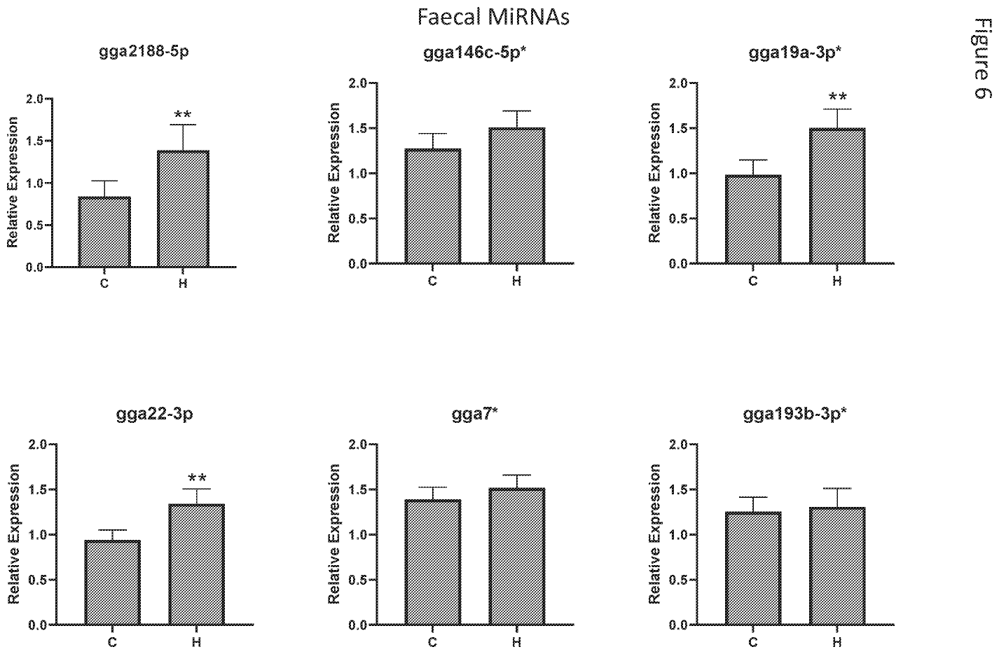

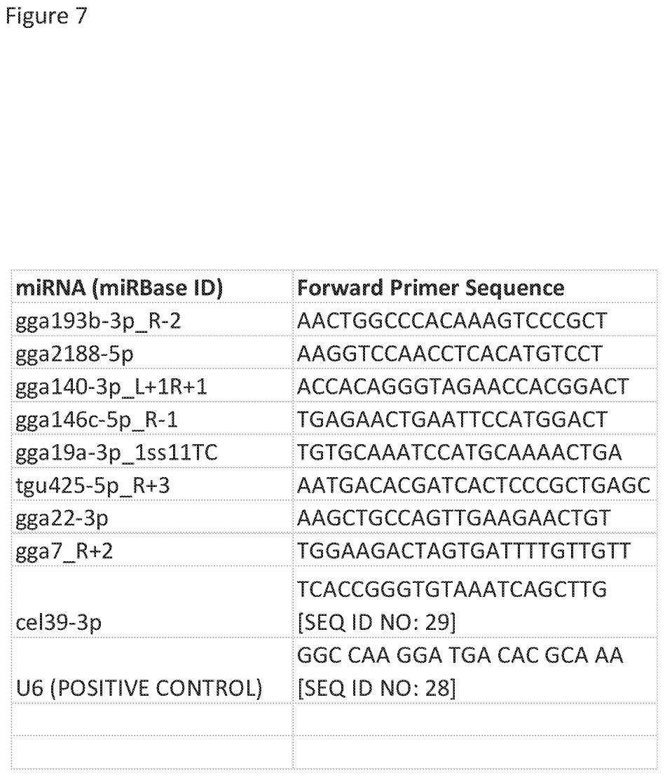

To demonstrate that miRNAs could also be extracted and amplified from faecal content samples, and are reflective of differentially regulated miRNAs in the caecal content, we performed qPCR assays (FIG. 6) for 6 of the differentially regulated miRNAs identified from caecal content qPCR validation. This showed that three of the six miRNAs were significantly upregulated in infected high lesion score birds compared to uninfected controls. These included gga2188-5p (1.6 fold increased expression, P<0.01), gga19a-3p_1ss11TC (1.3 fold increased expression, P<0.01), and gga22-3p (1.4 fold increased expression, P<0.01).

### Example 6 Discussion

These data demonstrate that it is possible to isolate and sequence miRNAs from chicken caecal and faecal content, demonstrating that faecal content could be used to non-invasively assess avian intestinal disease in a diagnostic capacity. Of the 95 differentially expressed miRNAs identified through sequencing, 19 were identified that were statistically significantly altered in infected versus uninfected controls. However, these did not further differentiate between high and low lesion scores in infection. 8 selected miRNAs were tested further by qPCR for validation, and 5 of these were shown to be significantly altered in high lesion score birds in biological replicates from a separate experiment, compared to uninfected controls and in some cases low lesion score birds. Finally, we tested these 8 miRNA candidates in the faeces of further biological replicates in a separate experiment. This showed 3 miRNAs to be significantly upregulated in faeces from high lesion score infected birds, and that the extraction techniques and qPCR methodology can successfully be applied to chicken faeces as a potential diagnostic sample.

Interestingly, one of these selected miRNA candidates (gga-miR-2188-5p) has also been shown to be upregulated in the small intestinal tissue of chickens infected with Eimeria necatrix (T. L. Liu et al., 2020). These miRNAs showed large fold changes in the caecal content of infected birds, and may alone, or in combination with gga19a-3p or gga22-3p form the basis of a non-invasive diagnostic faecal test for active E. tenella infection without the need for culling birds to perform post-mortem diagnosis. Further functional analysis demonstrated that there was significant over representation of the Mucin type O-Glycan biosynthesis pathway (adjusted P value of 0.0016), showing 6 hits for gene targets GALNT16, GALNT6, GALNT12, GALNT14, GALNT5, GCNT4. Of these differentially regulated genes, GALNT5 showed co-regulation by more than one miRNA (namely gga-miR-193b and gga-miR-128-3p). Interestingly, the Mucin type O-Glycan biosynthesis pathway has been shown to be differentially regulated by miRNAs in in nasal mucosa of human patients with chronic rhinosinusitis (Xuan, et al. 2019). It was speculated in this study that there was induction of goblet cell hyperplasia with chronicity which increased mucus layer production and exacerbated favourable growth conditions for pathogens. Intestinal mucus plays an important part in host-pathogen interactions. Intestinal mucus, is also rich in Mucin-type O-glycans and has been shown to form a critical protective layer between the intestinal lumen and the epithelial monolayer (Bergstrom and Xia 2013). In the context of Eimeria tenella infection, it has been shown that there is adherence of chicken intestinal mucins to the parasite which inhibits invasion in vitro (Tierney, et al. 2007). This suggests that possible differential mucin synthesis would likely influence Eimeria tenella infection. Further studies may be able to further elucidate the biological interactions of differentially regulated miRNAs identified herein, further refine their natural variation of expression in the faeces of uninfected and Eimeria infected birds for diagnostic test development, and further define their sensitivity and specificity for different Eimeria species intestinal infections.

### Example 7 Materials and Methods

Animal ethics statement. The work described was conducted in accordance with UK Home Office regulations under the Animals (Scientific Procedures) Act 1986 (ASPA). Protocols were approved by the Royal Veterinary College Animal Welfare and Ethical Review Body (AWERB). Study birds were observed twice per day for signs of illness and/or welfare impairment and were sacrificed under Home Office licence by cervical dislocation. Throughout the study all chickens had access to feed and water ad-libitum.

Parasite propagation. Sporulated E. tenella parasites of the Houghton reference strain were propagated and maintained as described previously (Long, Millard, Joyner & Norton, 1976) using specific pathogen free (SPF) Lohmann Valo chickens accommodated in ammonia-fumigated facilities. Chickens were received when 21 days old, infected seven days later by oral gavage and culled for parasite harvest when 35 days old.

### Animal Studies

Caecal content samples for initial qPCR validation of miRNA recovery and quality. Lohmann Valo chickens (28 days of age) reared under SPF conditions were infected by oral inoculation of 4,000 E. tenella oocysts or sham inoculated as part of a separate study. Caecal contents were collected seven days after inoculation from infected (n=6) and sham (n=3) chickens during post-mortem, immediately after cervical dislocation. Samples were stored at −80° C.

Caecal content samples for miRNA sequencing. Samples were taken as part of a larger study where 250, day-old, Cobb500 broiler chickens were housed in coccidia-free conditions (Macdonald, et al. 2017). At 21 days of age, 25 chickens in group 1 (uninfected control group) received a single inoculum of 1 ml of DNase/RNase-free water. In parallel, 225 broilers in group 2 (infected group) were inoculated with 35,000 sporulated E. tenella oocysts in 1 ml of water. Four and a half days (108 h) post infection all birds were culled (26 days old). Post-mortem, caecal tissue was assessed immediately for lesions and scored following a well established method (Johnson & Reid, 1970). Lesions were scored from 0 to 4:0 (no lesions), 1 (mild lesions), 2 (moderate lesions), 3 (severe lesions), 4 (very severe lesions). Caecal contents were collected and snap frozen in liquid nitrogen. All samples were stored at −80° C. until further processing. Caecal content samples were selected from female chickens that were uninfected controls (n=3), infected lesion score 0 (n=3) and infected lesion score 4 (n=3).

Caecal content samples for qPCR of selected miRNA targets: validation of RNAseq data in biological replicates. A second set of samples were collected from a duplicate experiment run 20 months after the first, providing independent biological replication. Nine samples were chosen, including uninfected controls (n=3), infected lesion score 0/1 (n=3) and infected lesion score 4 (n=3). All samples were stored at −80° C. until further processing.

### RNA Extraction and Preparation.

RNA was extracted from caecal content samples using the Norgen Stool Total RNA Purification Kit (Cat. No. 49500, Biotek, Canada) as per the manufacturer's instructions. Briefly, 200 mg of caecal content from each sample was homogenised using a Minibead Beater, (Biospec, Oklahoma) at 3.5×1000 oscillations per minute for 1 minute and a rapid spin column procedure was then used to extract and purify total RNA. An additional step of on-column DNA removal was conducted as per the manufacturer's instructions. The extracted total RNA samples were analysed using a Nanodrop spectrophotometer (NanoDrop, ND-1000 Spectrophotometer, North Carolina). Data were analysed using GraphPad Prism 7 software.

RNA extraction for method development was additionally performed using the mirVana miRNA Isolation Kit (Cat. No. AM 1560, Lot 00360891, Mirvana, ThermoFisher Scientific, Massachusetts) following the kit protocol, with minor modifications. 1 ml of Mirvana Lysis/Binding Solution was used per sample. 500 μl of the bead beaten sample was transferred to an RNase-free microcentrifuge tube with 50 μl of miRNA homogenate Additive. At the total RNA isolation stage, 500 μl of 100% (v/v) ethanol was added to the separated aqueous phase. Sample mixtures were added to the filter cartridge in two aliquots of 450 μl and centrifuged at 10000×g for 15 seconds between each addition. The filter cartridge and collection tube were centrifuged twice further, for 15 seconds both times. Samples were centrifuged for 10 seconds following the addition of both Wash Solution 1 and Wash Solution 2/3, and for 30 seconds following the Elution Solution RNaseZAP (Cat. No. R2020, Sigma-Aldrich, UK), an RNase decontamination solution, was used on equipment and work areas prior to experimentation with extracted RNAs to reduce RNase dependent RNA degradation.

Bioanalyzer. An Agilent RNA 6000 Nano Kit was used with an Agilent 2100 Electrophoresis Bioanalyzer to analyse the RNA samples. All sample concentrations were diluted to the recommended range of 25-500 ng/μl. The chip priming station, gel-dye mix preparation and loading the marker, ladder and samples were performed following manufacturer's instructions.

Polyadenylation and reverse transcription. cDNA was prepared using a Mir-X miRNA First-Strand Synthesis Kit (Cat. No. 638313, Clontech, France). The RNA samples were diluted using bottled ultrapure diethylpyrocarbonate (DEPC)-treated water to a standard concentration of 0.28 μg/μl. Appropriate reagents from the kits were added following the protocol and the mixtures were incubated in a thermocycler (G Storm Thermal Cycler with GS0096 96 well block, GT 11584) for 1 hour at 37° C., then for 5 minutes at 85° C. The product was made up to a final volume of 100 μl and used in qPCR.

Quantitative PCR (qPCR)

Initially, qPCR was performed using the SYBR Advantage qRT-PCR Kit (Cat. No. 638313, Clontech, France) following the given protocol. Each sample well contained 9 μl of RNase-free water, 12.5 μl SYBR Advantage Premix, 0.5 μl ROX Dye, 0.5 μl miRNA-specific primer, 0.5 μl mRQ 3′ primer and finally 2 μl of cDNA, to give a total volume of 25 μl per well. During the initial assessment of miRNA recovery and quality a panel of four primers were utilised (Table 1), chosen based on the previous study performed by Liu et al. (2016). qPCR amplification using U6 from the kit, a universal primer, was also performed for each cDNA sample to serve as a normalisation standard. All samples, U6 and no template controls (NTC) were performed in duplicate. Amplification was performed on a real-time qPCR instrument (CFX-96 new generation Real-Time PCR detection, C-1000 Thermal Cycler). Reactions were denatured for 10 seconds at 95° C., followed by 40 qPCR cycles consisting of 95° C. for 5 seconds and 60° C. for 20 seconds. Finally, a dissociation curve of 95° C. for 60 seconds, 55° C. for 5 seconds and 95° C. for 5 seconds was performed.

Subsequently, qPCR to validate the results of miRNA sequencing using independent biological replicates followed the same protocol, modified to include new target-specific primers. Here, nine apparently differentially regulated miRNA sequences were selected from the sequencing results. Primers were designed using miRbase (University of Manchester) and synthesised by Sigma-Aldrich. The entire sequence of the miRNA was used as the miRNA-specific, 5′ primer. For relative quantification, U6 snRNA supplied with the kit was used as a positive control using the ddCt method. All samples were run in duplicate. Statistical comparisons were made by ANOVA with Tukey's post hoc analysis.

Data Analysis. qPCR data was analysed using the Delta-Delta C, Method (ΔΔCt) (Livak & Schmittgen, 2001) and GraphPad Prism 7 software.

miRNA Sequencing, Read Processing and Quality Control

RNA samples were processed by LC Sciences, Houston, USA to generate a small RNA library using the Illumina Truseq™ Small RNA Preparation kit according to manufacturer protocols. Purified cDNA libraries were used for cluster generation on an Illumina Cluster Station and then sequenced on an Illumina HiSeq platform. Raw sequencing reads (50 nt) were obtained using Illumina's Sequencing Control Studio software version 2.8 (SCS v2.8) following real-time sequencing image analysis and base-calling by Illumina's Real-Time Analysis version 1.8.70 (RTA v1.8.70).

A proprietary pipeline script, ACGT101-miR v4.2 (LC Sciences), was used for sequencing data analysis. After the raw sequence reads, sequences were extracted from image data, a series of digital filters were applied to exclude various un-mappable sequencing reads. During data combination and analysis, low read sequences were removed. The small RNA sequences generated were mapped against pre-miRNA (mir) and mature miRNA (miR) listed sequences in miRBase (ftp://mirbase.org/pub/mirbase/CURRENT/, version 21) based on the public releases for Gallus gallus and other listed avian species. Sequences were also mapped against the G. gallus genome (ftp://ftp.ncbi.nlm.nih.gov/genomes/Gallus gallus/, version 4). Normalization of sequence counts in each sample (or data set) was achieved by dividing the counts by a library size parameter from the corresponding sample. The library size parameter was a median value of the ratio between the counts of a specific sample and a pseudo-reference sample. A count number in the pseudo-reference sample was the count geometric mean across all samples.

1. A method for determining the presence of an Eimeria Tenella intestinal infection in one or more test birds
wherein the method comprises determining the level of any 1, 2, 3, 4, 5, 6, 7, 8, 9, 10, 11, 12, 13, 14, 15, 16, 17, 18, or 19, or all of the following miRNAs in a faecal and/or caecal sample obtained from the one or more test birds:

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |
| [SEQ ID NO: 9] | gga-let-7c-5p* | TGAGGTAGTAGGTTGTATAGTTT |
| [SEQ ID NO: 10] | gga-miR-142-5p | CCCATAAAGTAGAAAGCACTAC |
| [SEQ ID NO: 11] | gga-miR-23b-3p* | ATCACATTGCCAGGGATTTCC |
| [SEQ ID NO: 12] | gga-miR-7b* | TGGAAGACTAGTGATTTTTGTTGT |
| [SEQ ID NO: 13] | gga-miR-30b-5p | TGTAAACATCCTACACTCAGCT |
| [SEQ ID NO: 14] | gga-miR-219b | CACAAGAATTGCGTTTGGACAA |
| [SEQ ID NO: 15] | tgu-miR-363-3p | AATTGCACGGTATCCATCTGT |
| [SEQ ID NO: 16] | gga-miR-128-3p | TCACAGTGAACCGGTCTCTTT |
| [SEQ ID NO: 17] | gga-miR-19b-3p* | TGTGCAAATCCATGCAAAACTGT |
| [SEQ ID NO: 18] | gga-miR-101-3p* | GTACAGTACTGTGATAACTGA |
| [SEQ ID NO: 19] | gga-miR-183* | TATGGCACTGGTAGAATTCACT. |

2. A method for determining the presence of an intestinal infection in one or more test birds wherein the intestinal infection is caused by an infectious agent,
wherein the method comprises determining the level of one or more miRNAs in a faecal and/or caecal sample obtained from the one or more test birds, wherein the level of the one or more miRNAs represents a miRNA signature indicative of the presence of said intestinal infection.

3. A method for determining the presence of a pathologically significant intestinal infection or a pathologically significant intestinal infection that requires treatment in one or more test birds, wherein the intestinal infection is caused by an infectious agent,
wherein the method comprises determining the level of one or more miRNAs in a faecal and/or caecal sample obtained from the one or more test birds, wherein the level of the one or more miRNAs represents a miRNA signature indicative of the presence of said pathologically significant intestinal infection or pathologically significant intestinal infection that requires treatment.

4. The method of any of claims 1-3 wherein determining the level of one or more miRNAs in the sample does not require isolation or purification or membrane vesicles, such as microvesicles or exosomes.

5. The method according to any of claims 2-4 wherein the infectious agent is an avian intestinal parasite, optionally is:
a) Eimeralla sp;
b) selected from Eimeria Tenella, Eimeria necatrix, Eimeria acervuline, Eimeria brunetti, Eumeria maxima, Eimeria mitis, Eimeria praecox;
c) is Eimeria tenella or Eimeria necatrix; or
d) Eimeria tenella

6. The method according to any of claims 2-4 wherein the infectious agent is a bacteria, optionally is Salmonella sp. or E. coli.

7. The method according to any of claims 1-6 wherein the one or more test birds is a chicken or a turkey, optionally is a chicken (Gallus gallus).

8. The method according to any of claims 1-7 wherein the one or more miRNAs are produced by the test bird, optionally produced by the intestinal tissue of the test bird.

9. The method according to any of the preceding claims wherein the method further comprises comparing the level of the one or more miRNAs to the level of the same one or more miRNAs in one or more control samples.

10. The method according to claim 9 wherein the control sample is a negative control faecal and/or caecal sample obtained from one or more birds that are not infected with the infectious agent, or are not infected with a pathologically relevant infection or a pathologically significant intestinal infection that requires treatment,
and wherein the test bird is determined to be infected with the infectious agent optionally infected with a pathologically significant infection or a pathologically significant intestinal infection that requires treatment, if the level of the one or more miRNAs in the faecal and/or caecal sample from the test bird is:
a) higher than the level of the one or more miRNAs in the negative control sample; and/or
b) above a predetermined range or within a pre-determined threshold level.

11. The method according to claim 10 wherein the test bird is determined to not be infected or to not be infected with a pathologically significant infection or a pathologically significant intestinal infection that requires treatment where the level of the one or more miRNAs in the test sample is:
a) the same as or substantially similar to the level of the same miRNAs in the negative control sample; and/or
b) within a predetermined range or below a predetermined threshold.

12. The method according to claim 9-11 wherein the control sample is a positive control faecal and/or caecal sample obtained from one or more birds that are infected with the infectious agent, or are infected with a pathologically relevant infection or a pathologically significant intestinal infection that requires treatment,
and wherein the test bird is determined to be infected with the infectious agent optionally infected with a pathologically significant infection or a pathologically significant intestinal infection that requires treatment, if the level of the one or more miRNAs in the faecal and/or caecal sample from the test subject is:
a) the same as or higher than the level of the same one or more miRNAs in the positive control sample; and/or
b) within a predetermined range of the level of the one or more miRNAs in the positive control sample.

13. The method according to claim 9-12 wherein the test subject is determined to not be infected or to not be infected with a pathologically significant infection or a pathologically significant intestinal infection that requires treatment where the level of the one or more miRNAs in the test sample is:
a) below or substantially below the level of the same one or more miRNAs in the positive control samples; and/or
b) below a predetermined lower threshold value or range of the one or more miRNAs in the positive control samples.

14. The method according to any of claims 2-13 wherein the method comprises determining the level of any 1, 2, 3, 4, 5, 6, 7, 8, 9, 10, 11, 12, 13, 14, 15, 16, 17, 18, or 19, or all of the following miRNAs:

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |
| [SEQ ID NO: 9] | gga-let-7c-5p* | TGAGGTAGTAGGTTGTATAGTTT |
| [SEQ ID NO: 10] | gga-miR-142-5p | CCCATAAAGTAGAAAGCACTAC |
| [SEQ ID NO: 11] | gga-miR-23b-3p* | ATCACATTGCCAGGGATTTCC |
| [SEQ ID NO: 12] | gga-miR-7b* | TGGAAGACTAGTGATTTTTGTTGT |
| [SEQ ID NO: 13] | gga-miR-30b-5p | TGTAAACATCCTACACTCAGCT |
| [SEQ ID NO: 14] | gga-miR-219b | CACAAGAATTGCGTTTGGACAA |
| [SEQ ID NO: 15] | tgu-miR-363-3p | AATTGCACGGTATCCATCTGT |
| [SEQ ID NO: 16] | gga-miR-128-3p | TCACAGTGAACCGGTCTCTTT |
| [SEQ ID NO: 17] | gga-miR-19b-3p* | TGTGCAAATCCATGCAAAACTGT |
| [SEQ ID NO: 18] | gga-miR-101-3p* | GTACAGTACTGTGATAACTGA |
| [SEQ ID NO: 19] | gga-miR-183* | TATGGCACTGGTAGAATTCACT |

or of miRNA with a sequence that has at least 90%, 91%, 92%, 93%, 94%, 95%, 96%, 97%, 98%, 99% or 100% sequence identity to the one or more miRNAs of SEQ ID NO: 1-19.

15. The method according to any of claims 1-14 wherein the method comprises determining the level of any 1, 2, 3, 4, 5, 6, 7 or 8 of the following miRNAs:

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

or of miRNA with a sequence that has at least 90%, 91%, 92%, 93%, 94%, 95%, 96%, 97%, 98%, 99% or 100% sequence identity to the one or more miRNAs of SEQ ID NO: 1-8.

16. The method according to any of claims 1-15 wherein the method comprises determining the level of any 1, 2, 3, 4, or 5 of the following miRNAs:

| | | |
|---|---|---|
| [SEQ ID NO: 2] | gga-miR-2188-5p | AAGGTCCAACCTCACATGTCCT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

or of miRNA with a sequence that has at least 90%, 91%, 92%, 93%, 94%, 95%, 96%, 97%, 98%, 99% or 100% sequence identity to the one or more miRNAs of SEQ ID NO: 2, 4, 5, 7 or 8.

17. The method according to any of claims 1-16 wherein the method comprises determining the level of any 1, 2 or 3 of the following miRNAs:

| [SEQ ID NO: 2] | |
|---|---|
| gga-miR-2188-5p | AAGGTCCAACCTCACATGTCCT |
| [SEQ ID NO: 5] | |
| gga-miR-19a-3p* | TGTGCAAATCCATGCAAAACTGA |
| [SEQ ID NO: 7] | |
| gga-miR-22-3p | AAGCTGCCAGTTGAAGAACTGT |

or of miRNA with a sequence that has at least 90%, 91%, 92%, 93%, 94%, 95%, 96%, 97%, 98%, 99% or 100% sequence identity to the one or more miRNAs of SEQ ID NO: 2, 5 or 7.

18. The method according to any one of claims 14-17 wherein the method comprises determining the level of:
a) any 1, 2, 3, 4, 5, 6, 7, 8, 9, 10, 11, 12, 13, 14, 15, 16, 17, 18, or 19, or all of the following miRNAs:

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |
| [SEQ ID NO: 9] | gga-let-7c-5p* | TGAGGTAGTAGGTTGTATAGTTT |
| [SEQ ID NO: 10] | gga-miR-142-5p | CCCATAAAGTAGAAAGCACTAC |
| [SEQ ID NO: 11] | gga-miR-23b-3p* | ATCACATTGCCAGGGATTTCC |
| [SEQ ID NO: 12] | gga-miR-7b* | TGGAAGACTAGTGATTTTTGTTGT |
| [SEQ ID NO: 13] | gga-miR-30b-5p | TGTAAACATCCTACACTCAGCT |
| [SEQ ID NO: 14] | gga-miR-219b | CACAAGAATTGCGTTTGGACAA |
| [SEQ ID NO: 15] | tgu-miR-363-3p | AATTGCACGGTATCCATCTGT |
| [SEQ ID NO: 16] | gga-miR-128-3p | TCACAGTGAACCGGTCTCTTT |
| [SEQ ID NO: 17] | gga-miR-19b-3p* | TGTGCAAATCCATGCAAAACTGT |
| [SEQ ID NO: 18] | gga-miR-101-3p* | GTACAGTACTGTGATAACTGA |
| [SEQ ID NO: 19] | gga-miR-183* | TATGGCACTGGTAGAATTCACT |

b) any 1, 2, 3, 4, 5, 6, 7 or 8 of the following miRNAs:

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

c) any 1, 2, 3, 4, or 5 of the following miRNAs:

| [SEQ ID NO: 2] | |
|---|---|
| gga-miR-2188-5p | AAGGTCCAACCTCACATGTCCT |
| [SEQ ID NO: 4] | |
| gga-miR-146c-5p* | TGAGAACTGAATTCCATGGACT |
| [SEQ ID NO: 5] | |
| gga-miR-19a-3p* | TGTGCAAATCCATGCAAAACTGA |
| [SEQ ID NO: 7] | |
| gga-miR-22-3p | AAGCTGCCAGTTGAAGAACTGT |
| [SEQ ID NO: 8] | |
| gga-miR-7* | TGGAAGACTAGTGATTTTGTTGTT |

d) any 1, 2 or 3 of the following miRNAs:

| [SEQ ID NO: 2] | |
|---|---|
| gga-miR-2188-5p | AAGGTCCAACCTCACATGTCCT |
| [SEQ ID NO: 5] | |
| gga-miR-19a-3p* | TGTGCAAATCCATGCAAAACTGA |
| [SEQ ID NO: 7] | |
| gga-miR-22-3p | AAGCTGCCAGTTGAAGAACTGT. |

19. The method according to any of claims 1-18 wherein the method comprises determining the level of:
a) all of the following miRNAs:

| [SEQ ID NO: 2] | |
|---|---|
| gga-miR-2188-5p | AAGGTCCAACCTCACATGTCCT |
| [SEQ ID NO: 5] | |
| gga-miR-19a-3p* | TGTGCAAATCCATGCAAAACTGA |
| [SEQ ID NO: 7] | |
| gga-miR-22-3p | AAGCTGCCAGTTGAAGAACTGT. |

b) all of the following miRNAs:

| [SEQ ID NO: 2] | |
|---|---|
| gga-miR-2188-5p | AAGGTCCAACCTCACATGTCCT |
| [SEQ ID NO: 4] | |
| gga-miR-146c-5p* | TGAGAACTGAATTCCATGGACT |
| [SEQ ID NO: 5] | |
| gga-miR-19a-3p* | TGTGCAAATCCATGCAAAACTGA |
| [SEQ ID NO: 7] | |
| gga-miR-22-3p | AAGCTGCCAGTTGAAGAACTGT |
| [SEQ ID NO: 8] | |
| gga-miR-7* | TGGAAGACTAGTGATTTTGTTGTT |

c) all of the following miRNAs:

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

or
d) all of the following miRNAs:

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |
| [SEQ ID NO: 9] | gga-let-7c-5p* | TGAGGTAGTAGGTTGTATAGTTT |
| [SEQ ID NO: 10] | gga-miR-142-5p | CCCATAAAGTAGAAAGCACTAC |
| [SEQ ID NO: 11] | gga-miR-23b-3p* | ATCACATTGCCAGGGATTTCC |
| [SEQ ID NO: 12] | gga-miR-7b* | TGGAAGACTAGTGATTTTTGTTGT |
| [SEQ ID NO: 13] | gga-miR-30b-5p | TGTAAACATCCTACACTCAGCT |
| [SEQ ID NO: 14] | gga-miR-219b | CACAAGAATTGCGTTTGGACAA |
| [SEQ ID NO: 15] | tgu-miR-363-3p | AATTGCACGGTATCCATCTGT |
| [SEQ ID NO: 16] | gga-miR-128-3p | TCACAGTGAACCGGTCTCTTT |
| [SEQ ID NO: 17] | gga-miR-19b-3p* | TGTGCAAATCCATGCAAAACTGT |
| [SEQ ID NO: 18] | gga-miR-101-3p* | GTACAGTACTGTGATAACTGA |
| [SEQ ID NO: 19] | gga-miR-183* | TATGGCACTGGTAGAATTCACT |

20. The method according to any of claims 1-19 wherein said level of the one or more miRNAs is determined using reverse transcription followed by qPCR, optionally wherein the following primers are used to determine the level of said miRNA:
Where the miRNA is [SEQ ID NO: 1] gga-miR-2188-5p, the forward primer is AAGGTCCAACCTCACATGTCCT [SEQ ID NO: 20] and the reverse primer is the Universal mRQ 3′ Primer supplied Mir-X™ miRNA First-Strand Synthesis and SYBR® qRT-PCR kit;
Where the miRNA is [SEQ ID NO: 2] gga19a-3p*, the forward primer is TGTGCAAATCCATGCAAAACTGA [SEQ ID NO: 21] and the reverse primer is the Universal mRQ 3′ Primer supplied Mir-X™ miRNA First-Strand Synthesis and SYBR® qRT-PCR kit;
Where the miRNA is [SEQ ID NO: 3] gga-miR-22-3p, the forward primer is AAGCTGCCAGTTGAAGAACTGT [SEQ ID NO: 22] and the reverse primer is the Universal mRQ 3′ Primer supplied Mir-X™ miRNA First-Strand Synthesis and SYBR® qRT-PCR kit;
Where the miRNA is [SEQ ID NO: 4] gga7*, the forward primer is TGGAAGACTAGTGATTTTGTTGTT [SEQ ID NO: 23] and the reverse primer is the Universal mRQ 3′ Primer supplied Mir-X™ miRNA First-Strand Synthesis and SYBR® qRT-PCR kit;
Where the miRNA is [SEQ ID NO: 5] gga146c-5p*, the forward primer is TGAGAACTGAATTCCATGGACT [SEQ ID NO: 24] and the reverse primer is the Universal mRQ 3′ Primer supplied Mir-X™ miRNA First-Strand Synthesis and SYBR® qRT-PCR kit;
Where the miRNA is [SEQ ID NO: 6] gga193b-3p*, the forward primer is AACTGGCCCACAAAGTCCCGCT [SEQ ID NO: 25] and the reverse primer is the Universal mRQ 3′ Primer supplied Mir-X™ miRNA First-Strand Synthesis and SYBR® qRT-PCR kit;
Where the miRNA is [SEQ ID NO: 7] gga140-3p*, the forward primer is ACCACAGGGTAGAACCACGGACT [SEQ ID NO: 26] and the reverse primer is the Universal mRQ 3′ Primer supplied Mir-X™ miRNA First-Strand Synthesis and SYBR® qRT-PCR kit; and/or
Where the miRNA is [SEQ ID NO: 8] tgu425-5p*, the forward primer is AATGACACGATCACTCCCGCTGAGC [SEQ ID NO: 27] and the reverse primer is the Universal mRQ 3′ Primer supplied Mir-X™ miRNA First-Strand Synthesis and SYBR® qRT-PCR kit.

21. A method for determining a miRNA signature predictive of intestinal infection with an infectious agent in birds, optionally predictive of pathologically significant intestinal infection, or pathologically significant intestinal infection that requires treatment, wherein the method comprises:
a) providing at least:
a first plurality of faecal and/or caecal samples; and
a second plurality of faecal and/or caecal samples
wherein each of the faecal and/or caecal samples in the first plurality of faecal and/or caecal samples is taken from a different individual bird from a first population of birds that display a first infection phenotype and wherein each of the faecal and/or caecal samples in the second plurality of faecal and/or caecal samples is taken from a different individual bird from a second population of birds that display a second infection phenotype;
b) determining the relative amounts of a range of miRNAs in the first plurality of faecal and/or caecal samples and in the second plurality of faecal and/or caecal samples;
c) identifying miRNAs that are present in statistically different amounts between the first plurality of faecal and/or caecal samples level and the second plurality of faecal and/or caecal samples, and thereby identifying the miRNA signature predictive of infection with the infectious agent, optionally predictive of pathologically significant intestinal infection or pathologically significant intestinal infection that requires treatment.

22. The method according to claim 21 wherein the first infection phenotype is “uninfected” or “no pathologically significant level of infection” or “no pathologically significant level of infection that requires treatment” and the second infection phenotype is “infected” or “pathologically significant infection” or “pathologically significant level of infection that requires treatment”.

23. The method according to any of claim 21 or 22 wherein the infectious agent is a parasite, optionally is:
a) Eimeralla sp;
b) selected from Eimeria Tenella, Eimeria necatrix, Eimeria acervuline, Eimeria brunetti, Eumeria maxima, Eimeria mitis, Eimeria praecox;
c) is Eimeria tenella or Eimeria necatrix; or
d) Eimeria tenella.

24. A kit comprising one or more or all of the following:
a) Means to determine the level of at least one of the miRNAs associated with the presence of said avian intestinal parasite;
b) means for the extraction and purification of RNA from a caecal and/or faecal sample;
c) a control sample of RNA prepared from a faecal sample from subjects that do not comprise the intestinal parasite,
optionally wherein the means to determine the level of at least one of the miRNAs comprises:
A) at least one pair of primers are arranged so as to amplify at least one or more of the following miRNAs:
i)

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |
| [SEQ ID NO: 9] | gga-let-7c-5p* | TGAGGTAGTAGGTTGTATAGTTT |
| [SEQ ID NO: 10] | gga-miR-142-5p | CCCATAAAGTAGAAAGCACTAC |
| [SEQ ID NO: 11] | gga-miR-23b-3p* | ATCACATTGCCAGGGATTTCC |
| [SEQ ID NO: 12] | gga-miR-7b* | TGGAAGACTAGTGATTTTTGTTGT |
| [SEQ ID NO: 13] | gga-miR-30b-5p | TGTAAACATCCTACACTCAGCT |
| [SEQ ID NO: 14] | gga-miR-219b | CACAAGAATTGCGTTTGGACAA |
| [SEQ ID NO: 15] | tgu-miR-363-3p | AATTGCACGGTATCCATCTGT |
| [SEQ ID NO: 16] | gga-miR-128-3p | TCACAGTGAACCGGTCTCTTT |
| [SEQ ID NO: 17] | gga-miR-19b-3p* | TGTGCAAATCCATGCAAAACTGT |
| [SEQ ID NO: 18] | gga-miR-101-3p* | GTACAGTACTGTGATAACTGA |
| [SEQ ID NO: 19] | gga-miR-183* | TATGGCACTGGTAGAATTCACT |

ii)

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

iii)

| | | |
|---|---|---|
| [SEQ ID NO: 2] | gga-miR-2188-5p | AAGGTCCAACCTCACATGTCCT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

or
iv)

| | | |
|---|---|---|
| [SEQ ID NO: 2] | gga-miR-2188-5p | AAGGTCCAACCTCACATGTCCT |
| [SEQ ID NO: 5] | gga-miR-19a-3p* | TGTGCAAATCCATGCAAAACTGA |
| [SEQ ID NO: 7] | gga-miR-22-3p | AAGCTGCCAGTTGAAGAACTGT. |

and/or
B) one or more hybridisation probes able to specifically hybridise to one or more of the following miRNAs:
i)

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |
| [SEQ ID NO: 9] | gga-let-7c-5p* | TGAGGTAGTAGGTTGTATAGTTT |
| [SEQ ID NO: 10] | gga-miR-142-5p | CCCATAAAGTAGAAAGCACTAC |
| [SEQ ID NO: 11] | gga-miR-23b-3p* | ATCACATTGCCAGGGATTTCC |
| [SEQ ID NO: 12] | gga-miR-7b* | TGGAAGACTAGTGATTTTTGTTGT |
| [SEQ ID NO: 13] | gga-miR-30b-5p | TGTAAACATCCTACACTCAGCT |
| [SEQ ID NO: 14] | gga-miR-219b | CACAAGAATTGCGTTTGGACAA |
| [SEQ ID NO: 15] | tgu-miR-363-3p | AATTGCACGGTATCCATCTGT |
| [SEQ ID NO: 16] | gga-miR-128-3p | TCACAGTGAACCGGTCTCTTT |
| [SEQ ID NO: 17] | gga-miR-19b-3p* | TGTGCAAATCCATGCAAAACTGT |
| [SEQ ID NO: 18] | gga-miR-101-3p* | GTACAGTACTGTGATAACTGA |
| [SEQ ID NO: 19] | gga-miR-183* | TATGGCACTGGTAGAATTCACT |

ii)

| SEQ ID | miRbase ID | Sequence |
|---|---|---|
| [SEQ ID NO: 1] | gga-miR-193b-3p* | AACTGGCCCACAAAGTCCCGCT |
| [SEQ ID NO: 2] | gga-miR-2188-5p | AAGGTCCAACCTCACATGTCCT |
| [SEQ ID NO: 3] | gga-miR-140-3p* | ACCACAGGGTAGAACCACGGACT |
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 6] | tgu-miR-425-5p* | AATGACACGATCACTCCCGCTGAGC |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |

iii)

| [SEQ ID NO: 2] | gga-miR-2188-5p | AAGGTCCAACCTCACATGTCCT |
|---|---|---|
| [SEQ ID NO: 4] | gga-miR-146c-5p* | TGAGAACTGAATTCCATGGACT |
| [SEQ ID NO: 5] | gga-miR-19a-3p* | TGTGCAAATCCATGCAAAACTGA |
| [SEQ ID NO: 7] | gga-miR-22-3p | AAGCTGCCAGTTGAAGAACTGT |
| [SEQ ID NO: 8] | gga-miR-7* | TGGAAGACTAGTGATTTTGTTGTT |
| or | | |

iv)

| | | |
|---|---|---|
| [SEQ ID NO: 2] | gga-miR-2188-5p | AAGGTCCAACCTCACATGTCCT |
| [SEQ ID NO: 5] | gga-miR-19a-3p* | TGTGCAAATCCATGCAAAACTGA |
| [SEQ ID NO: 7] | gga-miR-22-3p | AAGCTGCCAGTTGAAGAACTGT. |

25. A kit comprising:
A) any one or more of the following primers:
[SEQ ID NO: 20]
Forward primer AAGGTCCAACCTCACATGTCCT
[SEQ ID NO: 21]
Forward primer TGTGCAAATCCATGCAAAACTGA
[SEQ ID NO: 22]
Forward primer AAGCTGCCAGTTGAAGAACTGT
[SEQ ID NO: 23]
Forward primer TGGAAGACTAGTGATTTTGTTGTT
[SEQ ID NO: 24]
Forward primer TGAGAACTGAATTCCATGGACT
[SEQ ID NO: 25]
Forward primer AACTGGCCCACAAAGTCCCGCT
[SEQ ID NO: 26]
Forward primer ACCACAGGGTAGAACCACGGACT
[SEQ ID NO: 27]
Forward primer AATGACACGATCACTCCCGCTGAGC;
And comprising
B) a suitable reverse primer, optionally a reverse primer that is the Universal mRQ 3′ Primer supplied Mir-X™ miRNA First-Strand Synthesis and SYBR® qRT-PCR kit.

26. The kit according to claim 25 comprising:
A)
[SEQ ID NO: 21]
Forward primer AAGCTGCCAGTTGAAGAACTGT;
[SEQ ID NO: 24]
Forward primer TGAGAACTGAATTCCATGGACT;
and
[SEQ ID NO: 26]
Forward primer ACCACAGGGTAGAACCACGGACT;
and
a suitable reverse primer, optionally a reverse primer that is the Universal mRQ 3′ Primer supplied Mir-X™ miRNA First-Strand Synthesis and SYBR® qRT-PCR kit;
B)
[SEQ ID NO: 21]
Forward primer AAGCTGCCAGTTGAAGAACTGT;
[SEQ ID NO: 24]
Forward primer TGAGAACTGAATTCCATGGACT;
[SEQ ID NO: 26]
Forward primer ACCACAGGGTAGAACCACGGACT;
[SEQ ID NO: 23]
Forward primer TGGAAGACTAGTGATTTTGTTGTT;
and
[SEQ ID NO: 27]
Forward primer AATGACACGATCACTCCCGCTGAGC;
and
a suitable reverse primer, optionally a reverse primer that is the Universal mRQ 3′ Primer supplied Mir-X™ miRNA First-Strand Synthesis and SYBR® qRT-PCR kit;
or
C)
[SEQ ID NO: 20]
Forward primer AAGGTCCAACCTCACATGTCCT;
[SEQ ID NO: 21]
Forward primer TGTGCAAATCCATGCAAAACTGA;
[SEQ ID NO: 22]
Forward primer AAGCTGCCAGTTGAAGAACTGT;
[SEQ ID NO: 23]
Forward primer TGGAAGACTAGTGATTTTGTTGTT;
[SEQ ID NO: 24]
Forward primer TGAGAACTGAATTCCATGGACT;
[SEQ ID NO: 25]
Forward primer AACTGGCCCACAAAGTCCCGCT;
[SEQ ID NO: 26]
Forward primer ACCACAGGGTAGAACCACGGACT;
and
[SEQ ID NO: 27]
Forward primer AATGACACGATCACTCCCGCTGAGC;
and
a suitable reverse primer, optionally a reverse primer that is the Universal mRQ 3′ Primer supplied Mir-X™ miRNA First-Strand Synthesis and SYBR® qRT-PCR kit.

27. A method for screening a population of birds the presence of an avian intestinal parasite wherein the method comprises determining the presence of an avian intestinal parasite according to any of the preceding claims wherein the sample is a sample of faecal matter taken from the avian environment,
optionally wherein the sample comprises a fecal matter from a number of individual birds from the avian environment,
optionally wherein the method for screening is performed at periodic intervals, optionally at intervals of 1 week, 2 weeks, 3 weeks, 4 weeks, 1 month, 2 months, 3 months, 4 months, 5 months or 6 months or more.


SEQUENCE LISTING (29 sequences)

SEQ ID NO: 1 - RNA, 22 nt, Gallus gallus
  source 1..22: mol_type = transcribed RNA
aactggcccacaaagtcccgct

SEQ ID NO: 2 - RNA, 22 nt, Gallus gallus
  source 1..22: mol_type = transcribed RNA
aaggtccaacctcacatgtcct

SEQ ID NO: 3 - RNA, 23 nt, Gallus gallus
  source 1..23: mol_type = transcribed RNA
accacagggtagaaccacggact

SEQ ID NO: 4 - RNA, 22 nt, Gallus gallus
  source 1..22: mol_type = transcribed RNA
tgagaactgaattccatggact

SEQ ID NO: 5 - RNA, 23 nt, Gallus gallus
  source 1..23: mol_type = transcribed RNA
tgtgcaaatccatgcaaaactga

SEQ ID NO: 6 - RNA, 25 nt, Gallus gallus
  source 1..25: mol_type = transcribed RNA
aatgacacgatcactcccgctgagc

SEQ ID NO: 7 - RNA, 22 nt, Gallus gallus
  source 1..22: mol_type = transcribed RNA
aagctgccagttgaagaactgt

SEQ ID NO: 8 - RNA, 24 nt, Gallus gallus
  source 1..24: mol_type = transcribed RNA
tggaagactagtgattttgttgtt

SEQ ID NO: 9 - RNA, 23 nt, Gallus gallus
  source 1..23: mol_type = transcribed RNA
tgaggtagtaggttgtatagttt

SEQ ID NO: 10 - RNA, 22 nt, Gallus gallus
  source 1..22: mol_type = transcribed RNA
cccataaagtagaaagcactac

SEQ ID NO: 11 - RNA, 21 nt, Gallus gallus
  source 1..21: mol_type = transcribed RNA
atcacattgccagggatttcc

SEQ ID NO: 12 - RNA, 24 nt, Gallus gallus
  source 1..24: mol_type = transcribed RNA
tggaagactagtgatttttgttgt

SEQ ID NO: 13 - RNA, 22 nt, Gallus gallus
  source 1..22: mol_type = transcribed RNA
tgtaaacatcctacactcagct

SEQ ID NO: 14 - RNA, 22 nt, Gallus gallus
  source 1..22: mol_type = transcribed RNA
cacaagaattgcgtttggacaa

SEQ ID NO: 15 - RNA, 21 nt, Gallus gallus
  source 1..21: mol_type = transcribed RNA
aattgcacggtatccatctgt

SEQ ID NO: 16 - RNA, 21 nt, Gallus gallus
  source 1..21: mol_type = transcribed RNA
tcacagtgaaccggtctcttt

SEQ ID NO: 17 - RNA, 23 nt, Gallus gallus
  source 1..23: mol_type = transcribed RNA
tgtgcaaatccatgcaaaactgt

SEQ ID NO: 18 - RNA, 21 nt, Gallus gallus
  source 1..21: mol_type = transcribed RNA
gtacagtactgtgataactga

SEQ ID NO: 19 - RNA, 22 nt, Gallus gallus
  source 1..22: mol_type = transcribed RNA
tatggcactggtagaattcact

SEQ ID NO: 20 - DNA, 22 bp, synthetic construct
  source 1..22: mol_type = other DNA
aaggtccaacctcacatgtcct

SEQ ID NO: 21 - DNA, 23 bp, synthetic construct
  source 1..23: mol_type = other DNA
tgtgcaaatccatgcaaaactga

SEQ ID NO: 22 - DNA, 22 bp, synthetic construct
  source 1..22: mol_type = other DNA
aagctgccagttgaagaactgt

SEQ ID NO: 23 - DNA, 24 bp, synthetic construct
  source 1..24: mol_type = other DNA
tggaagactagtgattttgttgtt

SEQ ID NO: 24 - DNA, 22 bp, synthetic construct
  source 1..22: mol_type = other DNA
tgagaactgaattccatggact

SEQ ID NO: 25 - DNA, 22 bp, synthetic construct
  source 1..22: mol_type = other DNA
aactggcccacaaagtcccgct

SEQ ID NO: 26 - DNA, 23 bp, synthetic construct
  source 1..23: mol_type = other DNA
accacagggtagaaccacggact

SEQ ID NO: 27 - DNA, 25 bp, synthetic construct
  source 1..25: mol_type = other DNA
aatgacacgatcactcccgctgagc

SEQ ID NO: 28 - DNA, 20 bp, synthetic construct
  source 1..20: mol_type = other DNA
ggccaaggatgacacgcaaa

SEQ ID NO: 29 - DNA, 22 bp, synthetic construct
  source 1..22: mol_type = other DNA
tcaccgggtgtaaatcagcttg